(19) United States
(12) Patent Application Publication
(10) Pub. No.: US 2026/0327239 A1
(43) Pub. Date: Oct. 22, 2026

(54) COMPOUNDS FOR COVALENT INHIBITION OF SHP2

(71) Applicant: Purdue Research Foundation, West Lafayette, IN (US)

(72) Inventors: Mingji Dai, Alpharetta, GA (US); Aaron Krabill, Arlington, MA (US); Weida Liang, San Diego, CA (US); Zhong-Yin Zhang, West Lafayette, IN (US)

(73) Assignee: Purdue Research Foundation, West Lafayette, IN (US)

(21) Appl. No.: 19/484,098

(22) PCT Filed: Jun. 3, 2024
(86) PCT No.: PCT/US2024/032193

§ 371 Date: Nov. 13, 2025

Related U.S. Application Data
(60) Provisional application No. 63/521,463, filed on Jun. 16, 2023.

(51) Int. Cl. C07D 491/107; A61K 31/16; A61K 31/19; A61K 31/357; A61K 31/397; A61K 31/404; A61K 31/4453; A61K 31/451; A61K 31/472; A61K 31/496; A61K 31/506; A61K 31/5377; A61K 31/553; C07C 62/38; C07C 235/82; C07D 209/42; C07D 211/38; C07D 211/52; C07D 217/06; C07D 239/42; C07D 267/18; C07D 295/116; C07D 295/185; C07D 319/18

(52) U.S. Cl. CPC C07D 491/107; A61K 31/16; A61K 31/19; A61K 31/357; A61K 31/397; A61K 31/404; A61K 31/4453; A61K 31/451; A61K 31/472; A61K 31/496; A61K 31/506; A61K 31/5377; A61K 31/553; C07C 62/38; C07C 235/82; C07D 209/42; C07D 211/38; C07D 211/52; C07D 217/06; C07D 239/42; C07D 267/18; C07D 295/116; C07D 295/185; C07D 319/18; C07C 2602/44

(57) ABSTRACT

Compounds that are covalent inhibitors of Src homology region 2 (SH2)-containing protein tyrosine phosphatase 2 (SHP2); compositions comprising same; and their use for treating cancers.

### CROSS-REFERENCE TO RELATED APPLICATIONS

This application claims priority to U.S. provisional patent application No. 63/521,463, which was filed Jun. 16, 2023, and which is hereby incorporated by reference in its entirety.

### STATEMENT OF GOVERNMENT SUPPORT

This invention was made with government support under GM128570, CA069202, and CA023168 awarded by the National Institutes of Health. The government has certain rights in the invention.

### TECHNICAL FIELD

The present disclosure relates to compounds that are covalent inhibitors of Src homology region 2 (SH2)-containing protein tyrosine phosphatase 2 (SHP2) and their use in inhibiting SHP2, such as their use in treating SHP2-associated cancers.

### BACKGROUND

This section introduces aspects that may help facilitate a better understanding of the disclosure. Accordingly, these statements are to be read in this light and are not to be construed as admissions about what is or is not prior art.

Protein tyrosine phosphatases (PTPs) are key regulators of signal transduction in cells that counteract the activity of protein tyrosine kinases to maintain proper levels of cellular tyrosine phosphorylation. Misregulated PTP function has been associated with many human diseases, including cancers, diabetes, and neurodegenerative and inflammatory disorders. As a bona fide oncogene, the Src homology domain-containing PTP 2 (SHP2, encoded by the PTPN11 gene) functions as a shared signaling node and is associated with a variety of cancers. Thus, SHP2 is an important anticancer therapeutic target.

Natural products play a vital role in the development of lifesaving drug molecules. Their structural novelty, diversity, and complexity make them unparalleled for novel therapeutic development. However, due to their structural complexity and natural scarcity, it is often difficult to conduct a comprehensive medicinal chemistry study around the active natural product to improve its potency, selectivity, and other drug properties. Natural products are also valuable sources of covalent protein modifiers due to the presence of electrophilic groups.

The application of natural products and their derivatives for the development of PTP inhibitors is an approach that has previously been shown to be fruitful. The utility of electrophilic natural products as novel and selective inhibitors of PTPs, including SHP2, has been demonstrated. The electrophilic group covalently modifies a target protein (ACS Omega 2020, 5, 2690-2698, and Int'l Pat. App. Pub. No. WO 2013/170770). The synthesized covalent inhibitors have several advantages, such as low dose requirement for achievable high potency, prolonged duration of action (due to covalent bond formation), and resistance to mutations. However, they lack selective inactivation of proteins, and off-target interactions lead to toxic side effects. Thus, only a few of the covalent drugs are approved by the FDA.

Therefore, there is an unmet need for a compound that covalently inhibits protein tyrosine phosphatase SHP2 with selective inactivation of the targeted protein. It is an object of the present disclosure to provide such a compound. This and other objects and advantages, as well as inventive features, will be apparent from the detailed description.

### SUMMARY

Provided is a compound of formula (I) or a pharmaceutically acceptable salt, hydrate, tautomer, or optical isomer thereof:

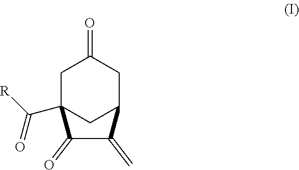

wherein
R is H, alkyl, alkenyl, alkynyl, cycloalkyl, acyl, heterocycloalkyl, aryl, heteroaryl, OR₁, or NR₁R₂, wherein R can be optionally substituted with at least one of —COOR₁, —SO₃R₁, halo, cyano, nitro, aryl, OR₁, and NR₁R₂;
wherein each R₁ and R₂ independently is H, alkyl, alkenyl, alkynyl, cycloalkyl, acyl, heterocycloalkyl, aryl, heteroaryl, OR₁, or NH₂; wherein each R₁ and R₂ can be optionally independently substituted with at least one of —COOR₁, —SO₃R₁, halo, cyano, nitro, cycloalkyl, aryl, heterocyclyl, OR₁, and NR₁R₂;
or
R₁ and R₂, together with the nitrogen atom to which they are attached, form a 4- to 8-membered heterocyclic ring, which contains one or more heteroatoms selected from O, N, and S and is optionally substituted with at least one of halo, alkyl, cycloalkyl, heterocycloalkyl, aryl, heteroaryl, —COOR₁, —SO₃R₁, cyano, OR₁, COR₁, and CF₃, wherein alkyl, cycloalkyl, heterocycloalkyl, aryl, and heteroaryl can be optionally further substituted with halo, cyano, or substituted or unsubstituted aryl, wherein aryl can be substituted with at least one of halo, alkyl, cyano, phenyl, OCH₃, and CF₃.

Provided is a compound of formula (IA) or a pharmaceutically acceptable salt, hydrate, tautomer, or optical isomer thereof:

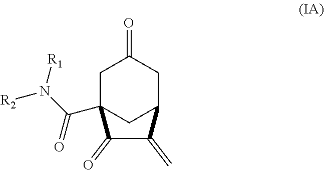

wherein each R₁ and R₂ independently is H, alkyl, alkenyl, alkynyl, cycloalkyl, acyl, heterocycloalkyl, aryl, heteroaryl, OR₁, or NH₂; wherein each R₁ and R₂ can be optionally independently substituted with at least one of —COOR₁, —SO₃R₁, halo, cyano, nitro, cycloalkyl, aryl, heterocyclyl, OR₁, and NR₁R₂;
or
R₁ and R₂, together with the nitrogen atom to which they are attached, form a 4- to 8-membered heterocyclic ring, which contains one or more heteroatoms selected from O, N, and S and is optionally substituted with at least one of halo, alkyl, cycloalkyl, heterocycloalkyl, aryl, heteroaryl, COOR₁, —SO₃R₁, cyano, OR₁, COR₁, and CF₃, wherein alkyl, cycloalkyl, heterocycloalkyl, aryl, and heteroaryl can be optionally further substituted with halo, cyano, or substituted or unsubstituted aryl, wherein aryl can be substituted with at least one of halo, alkyl, cyano, phenyl, OCH₃, and CF₃.

In some embodiments, the compound of formula (IA) is:

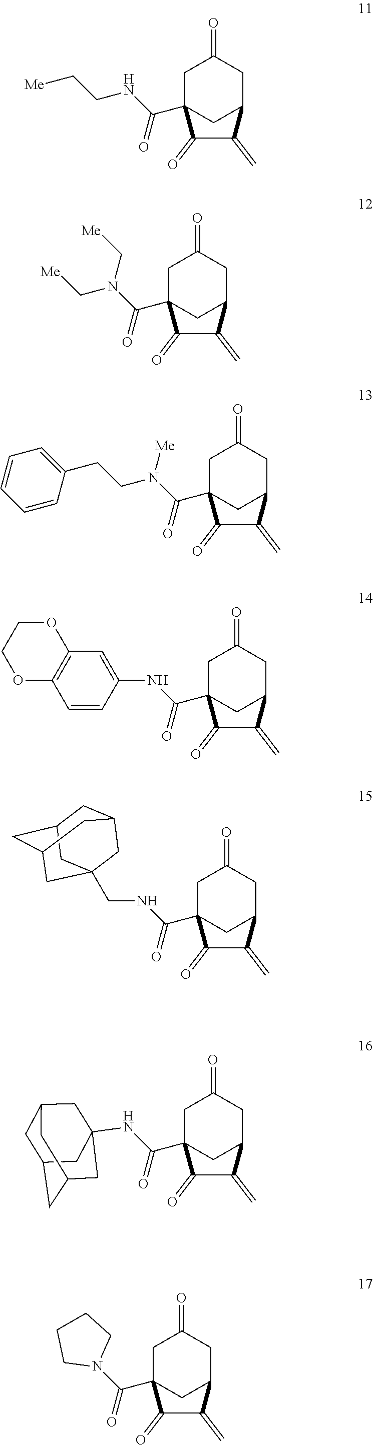

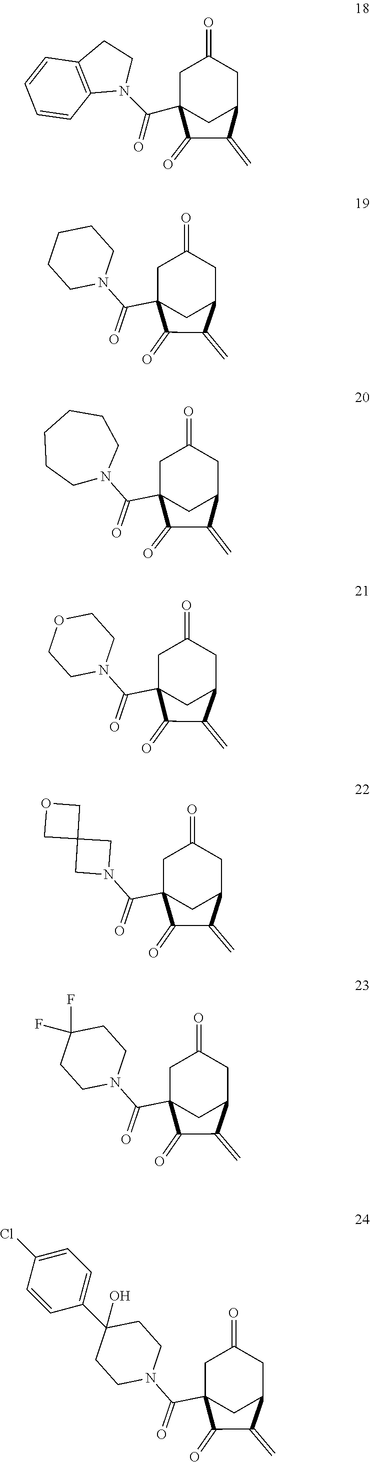

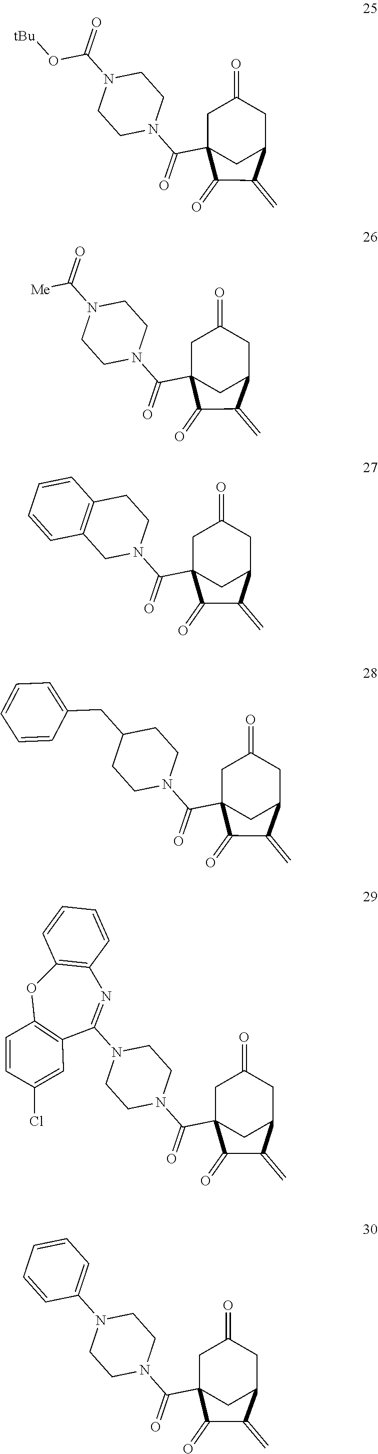 [SMILES: C=C1C(=O)[C@@]2(C(=O)N3CCN(C(=O)OC(C)(C)C)CC3)CC(=O)C[C@@H]1C2 ; C=C1C(=O)[C@@]2(C(=O)N3CCN(C(C)=O)CC3)CC(=O)C[C@@H]1C2 ; C=C1C(=O)[C@@]2(C(=O)N3CCc4ccccc4C3)CC(=O)C[C@@H]1C2 ; C=C1C(=O)[C@@]2(C(=O)N3CCC(Cc4ccccc4)CC3)CC(=O)C[C@@H]1C2 ; C=C1C(=O)[C@@]2(C(=O)N3CCN(C4=Nc5ccccc5Oc5ccc(Cl)cc54)CC3)CC(=O)C[C@@H]1C2 ; C=C1C(=O)[C@@]2(C(=O)N3CCN(c4ccccc4)CC3)CC(=O)C[C@@H]1C2]

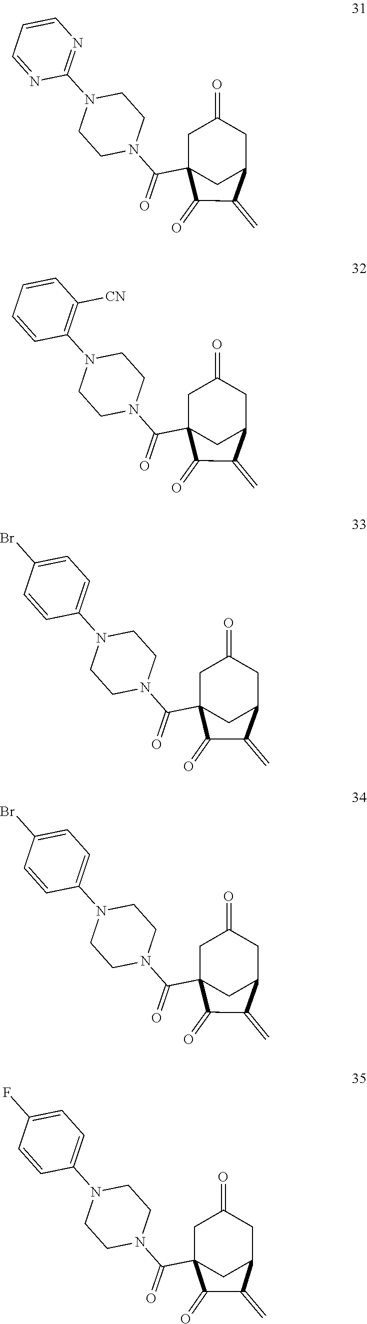 [SMILES: C=C1C(=O)[C@@]2(C(=O)N3CCN(c4ncccn4)CC3)CC(=O)C[C@@H]1C2 ; C=C1C(=O)[C@@]2(C(=O)N3CCN(c4ccccc4C#N)CC3)CC(=O)C[C@@H]1C2 ; C=C1C(=O)[C@@]2(C(=O)N3CCN(c4ccc(Br)cc4)CC3)CC(=O)C[C@@H]1C2 ; C=C1C(=O)[C@@]2(C(=O)N3CCN(c4ccc(Br)cc4)CC3)CC(=O)C[C@@H]1C2 ; C=C1C(=O)[C@@]2(C(=O)N3CCN(c4ccc(F)cc4)CC3)CC(=O)C[C@@H]1C2]

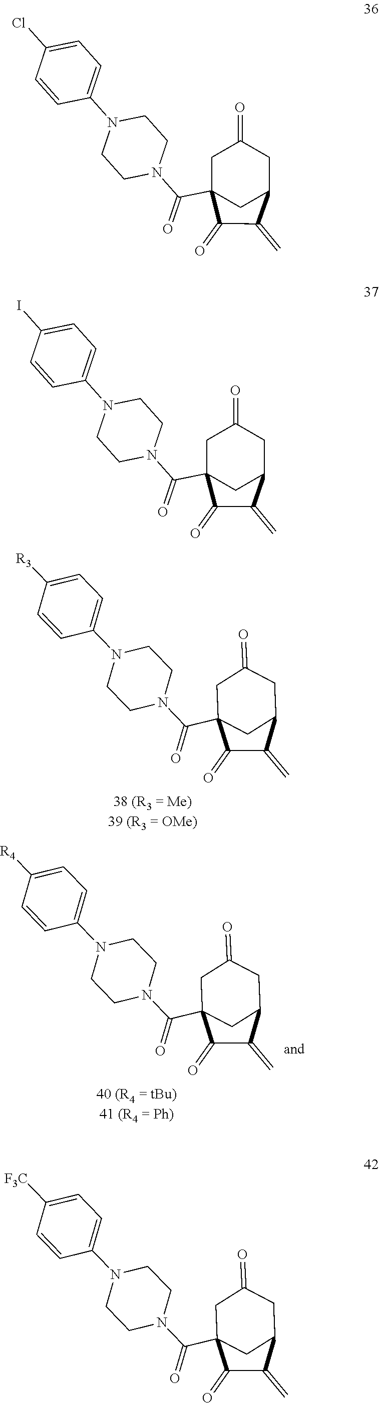

or a pharmaceutically acceptable salt, hydrate, tautomer, or optical isomer thereof.

Provided is a pharmaceutical composition comprising a compound of formula (I) or (IA), or a pharmaceutically acceptable salt, hydrate, tautomer, or optical isomer of either of the foregoing and a pharmaceutically acceptable carrier, excipient, or diluent.

Provided is a pharmaceutical composition comprising (i) a compound of formula (I) or (IA) or a pharmaceutically acceptable salt, hydrate, tautomer, or optical isomer of either of the foregoing, (ii) one or more additional therapeutic agents, and (iii) at least one pharmaceutically acceptable carrier, excipient, or diluent.

Further provided is a method of treating or inhibiting cancer in a patient, which method comprises administering to the patient a therapeutically effective amount of a compound of formula (I) or (IA) or a pharmaceutically acceptable salt, hydrate, tautomer, or optical isomer of either of the foregoing or a pharmaceutical composition comprising the compound and a pharmaceutically acceptable carrier, excipient, or diluent. In some embodiments, the compound can be administered orally or parenterally.

The cancer is mediated by an Src homology region 2 containing protein tyrosine phosphatase 2- (SHP2). In some embodiments, the cancer is leukemia or lung, breast, gastric, laryngeal, liver, or oral cancer.

Further provided is a method of inhibiting SHP2 in a patient, which method comprises administering to the patient a therapeutically effective amount of a compound of formula (I) or (IA) or a pharmaceutically acceptable salt, hydrate, tautomer, or optical isomer of either of the foregoing or a pharmaceutical composition comprising the compound and a pharmaceutically acceptable carrier, excipient, or diluent.

In some embodiments, the compound can be administered orally or parenterally.

Still further provided is a pharmaceutical combination comprising (i) a compound of formula (I) or (IA) or a pharmaceutically acceptable salt, hydrate, tautomer, or optical isomer of either of the foregoing, (ii) an additional therapeutic agent, and (iii) optionally at least one pharmaceutically acceptable carrier, excipient, or diluent.

### BRIEF DESCRIPTION OF THE DRAWINGS

The above and other objects, features, and advantages of the present disclosure will be apparent when the description is read in conjunction with the drawings.

FIG. 1A shows the efficacy of the halogen-substituted phenyl compounds correlated well with the presence of a hydrophobic group (Hansch Pi value).

FIG. 1B shows the efficacy of the halogen-substituted phenyl compounds correlated well with the presence of an electronegative group (Hammett Sigma value).

FIG. 2A shows the removal of the electrophile Michael acceptor carbonyl group from the compound (34) resulted in a complete loss of activity. This analog was inactive against Src homology region 2 (SH2)-containing protein tyrosine phosphatase 2 (SHP2), suggesting that an electrophilic moiety is required to inhibit and inactivate SHP2, supporting the proposed covalent mechanism of action for this compound.

FIG. 2B shows the active site of SHP2 was protected from inactivation by the presence of 500 μM vanadate (up-pointing triangles) upon dilution into substrate when compared to dimethylsulfoxide (DMSO; circles) or 500 μM vanadate alone (squares). Little residual activity was detected when SHP2 was incubated with compound (10) alone.

FIG. 3A shows the adduct formation on SHP2 by intact protein liquid chromatography-mass spectrometry (LC-MS). 20 μM phenyl vinyl sulfonate (PVSN) was treated with 10 μM SHP2 a major mass shift of 188 Da was observed with an overall 21% adduct formation on SHP2.

FIG. 3B shows the adduct formation of compound (37) on SHP2 by intact protein liquid chromatography-mass spectrometry (LC-MS). 10 μM compound (37) covalently modified with 10 μM SHP2 resulted in a mass shift of 461 Da, corresponding to the SHP2-37 adduct, with an overall adduct formation of 33%.

FIG. 4A shows the predicted covalent binding pose for compound (42) (sticks) in the active site of SHP2 (PDB ID 3O5X) (grey surface).

FIG. 4B shows hydrogen bonds are predicted to form between the bicyclic core of compound (42) and lysine (Lys) 366. Additional hydrogen bonds are predicted to form between the amide carbonyl of compound (42) and arginine (Arg) 465. A pi-pi interaction is predicted to occur between the phenyl ring of compound (42) and histidine (His) 426.

FIG. 4C shows the 4-substituted phenyl ring of compound (42) is predicted to interact with a nearby peripheral pocket of SHP2. The electrostatic surface potential of this pocket is positive.

FIG. 5 shows the 4-substituted phenyl ring of compound (37) is predicted to interact with a peripheral pocket of SHP2. Forming interactions with this pocket may provide additional selectivity for SHP2 over Src homology region 2 (SH2)-containing protein tyrosine phosphatase 1 (SHP1), Lymphoid-specific tyrosine phosphatase (LYP), and Protein tyrosine phosphatase-1B (PTP1B). Residues that imparted selectively towards SHP2 were His 426, glycine (Gly) 427, Glutamine (Gln 508), and Alanine (Ala) 509.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “covalent inhibitors” refers to inhibitors that form a covalent bond, which can be a strong covalent bond, with the target protein.

The term “SHP2” refers to Src homology region 2 (SH2)-containing protein tyrosine phosphatase 2 (SHP2, also known as SH2 domain-containing protein tyrosine phosphatase 2; SH-PTP2; SHP-2; BPTP3; PTP2C; protein tyrosine phosphatase non-receptor type 11 and PTPN11). SHP2 is encoded by the PTPN11 gene.

The terms “electrophile” and “electrophilic warhead” are used interchangeably.

The present disclosure is predicated, at least in part, on the role of natural products that can covalently modify cellular target proteins in developing lifesaving drug molecules. Many natural products contain diverse electrophilic warheads, which can react with a specific nucleophilic residue of targeted cellular proteins via covalent bond formation. The covalent inhibition of the targeted protein can potentially address targets with shallow, undruggable binding sites, therefore rendering those targets druggable. Further, covalent inhibition can provide high biochemical efficiency, which can translate to lower doses and reduced off-target effects; it can mitigate the development of drug resistance resulting from mutation of the binding site and reduce dosing frequency.

Studies revealed that natural products containing electrophilic warheads are selective inhibitors of PTPs including SHP2 (Davis, D. C. et al., J. Am. Chem. Soc. 2018, 140, 17465-17473). Ent-kaurene diterpenoids containing bicyclo[3.2.1]octane α-methylene ketone as the pharmacophore have significant biological activities, including anticancer and antiviral activity. However, their synthesis is very challenging with low yields.

In view of the above, the present disclosure provides compounds inspired by the complex and diverse ent-kaurene diterpenoids. The compounds comprise bicyclo[3.2.1]octane α-methylene ketone (A) as their pharmacophore.

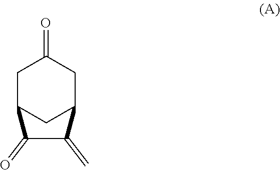

The compounds comprise electrophilic Michael acceptors, such as an α, β-unsaturated carbonyl group, which can form a covalent bond with a reactive functional group in the target enzyme, such as cysteine 459. The compounds can covalently inhibit SHP2, which is an important anticancer therapeutic target.

Provided is a compound of formula (I) or a pharmaceutically acceptable salt, hydrate, tautomer, or optical isomer thereof:

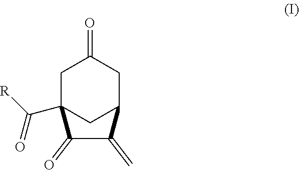

wherein
R is H, alkyl, alkenyl, alkynyl, cycloalkyl, acyl, heterocycloalkyl, aryl, heteroaryl, OR₁, or NR₁R₂, wherein R can be optionally substituted with at least one of —COOR₁, —SO₃R₁, halo, cyano, nitro, aryl, OR₁, and NR₁R₂;
wherein each R₁ and R₂ independently is H, alkyl, alkenyl, alkynyl, cycloalkyl, acyl, heterocycloalkyl, aryl, heteroaryl, OR₁, or NH₂; wherein each R₁ and R₂ can be optionally independently substituted with at least one of —COOR₁, —SO₃R₁, halo, cyano, nitro, cycloalkyl, aryl, heterocyclyl, OR₁, and NR₁R₂; or
R₁ and R₂, together with the nitrogen atom to which they are attached, form a 4- to 8-membered heterocyclic ring, which contains one or more heteroatoms selected from O, N, and S and is optionally substituted with at least one of halo, alkyl, cycloalkyl, heterocycloalkyl, aryl, heteroaryl, —COOR₁, —SO₃R₁, cyano, OR₁, COR₁, and CF₃, wherein alkyl, cycloalkyl, heterocycloalkyl, aryl, and heteroaryl can be optionally further substituted with halo, cyano, or substituted or unsubstituted aryl, wherein aryl can be substituted with at least one of halo, alkyl, cyano, phenyl, OCH₃, and CF₃.

Further provided is a compound of formula (IA) or a pharmaceutically acceptable salt, hydrate, tautomer, or optical isomer thereof:

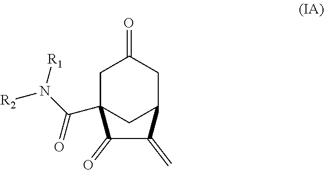

wherein
each R₁ and R₂ independently is H, alkyl, alkenyl, alkynyl, cycloalkyl, acyl, heterocycloalkyl, aryl, heteroaryl, OR₁, or NH₂; wherein each R₁ and R₂ can be optionally independently substituted with at least one of —COOR₁, —SO₃R₁, halo, cyano, nitro, cycloalkyl, aryl, heterocyclyl, OR₁, and NR₁R₂;
or
R₁ and R₂, together with the nitrogen atom to which they are attached, form a 4- to 8-membered heterocyclic ring, which contains one or more heteroatoms selected from O, N, and S and is optionally substituted with at least one of halo, alkyl, cycloalkyl, heterocycloalkyl, aryl, heteroaryl, COOR₁, —SO₃R₁, cyano, OR₁, COR₁, and CF₃, wherein alkyl, cycloalkyl, heterocycloalkyl, aryl, and heteroaryl can be optionally further substituted with halo, cyano, or substituted or unsubstituted aryl, wherein aryl can be substituted with at least one of halo, alkyl, cyano, phenyl, OCH, and CF.

In some embodiments, the compound of formula (TA) is:

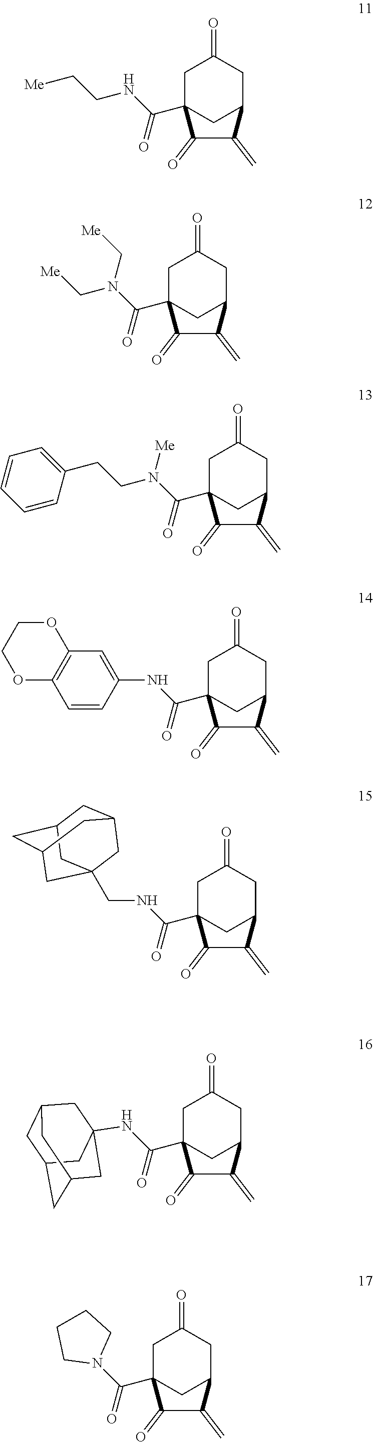

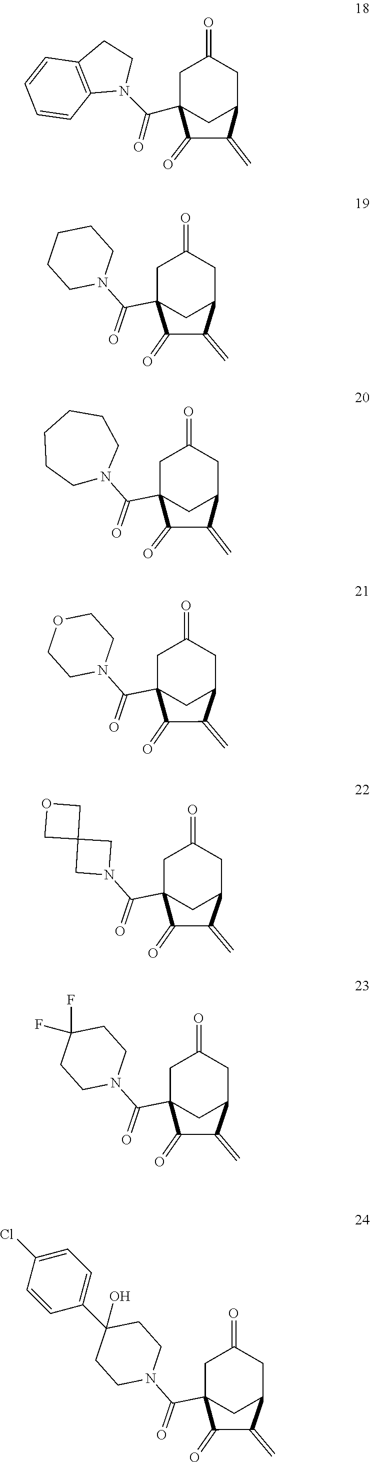

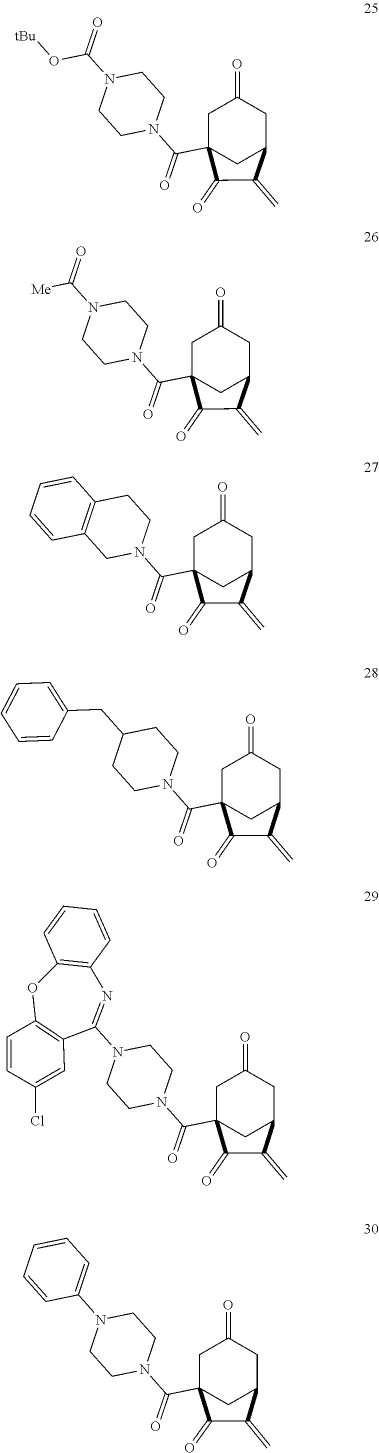 [SMILES: C=C1C(=O)[C@@]2(C(=O)N3CCN(C(=O)OC(C)(C)C)CC3)CC(=O)C[C@@H]1C2 ; C=C1C(=O)[C@@]2(C(=O)N3CCN(C(C)=O)CC3)CC(=O)C[C@@H]1C2 ; C=C1C(=O)[C@@]2(C(=O)N3CCc4ccccc4C3)CC(=O)C[C@@H]1C2 ; C=C1C(=O)[C@@]2(C(=O)N3CCC(Cc4ccccc4)CC3)CC(=O)C[C@@H]1C2 ; C=C1C(=O)[C@@]2(C(=O)N3CCN(C4=Nc5ccccc5Oc5ccc(Cl)cc54)CC3)CC(=O)C[C@@H]1C2 ; C=C1C(=O)[C@@]2(C(=O)N3CCN(c4ccccc4)CC3)CC(=O)C[C@@H]1C2]

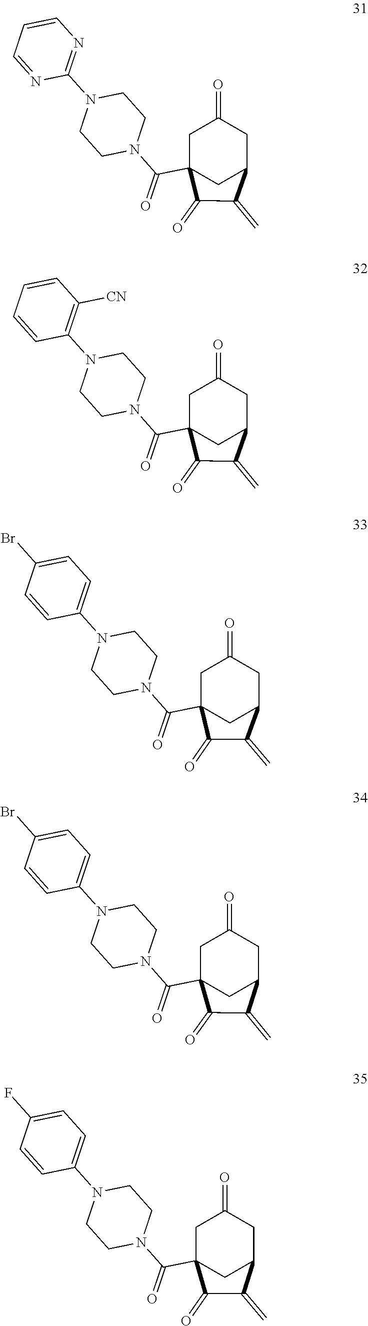 [SMILES: C=C1C(=O)[C@@]2(C(=O)N3CCN(c4ncccn4)CC3)CC(=O)C[C@@H]1C2 ; C=C1C(=O)[C@@]2(C(=O)N3CCN(c4ccccc4C#N)CC3)CC(=O)C[C@@H]1C2 ; C=C1C(=O)[C@@]2(C(=O)N3CCN(c4ccc(Br)cc4)CC3)CC(=O)C[C@@H]1C2 ; C=C1C(=O)[C@@]2(C(=O)N3CCN(c4ccc(Br)cc4)CC3)CC(=O)C[C@@H]1C2 ; C=C1C(=O)[C@@]2(C(=O)N3CCN(c4ccc(F)cc4)CC3)CC(=O)C[C@@H]1C2]

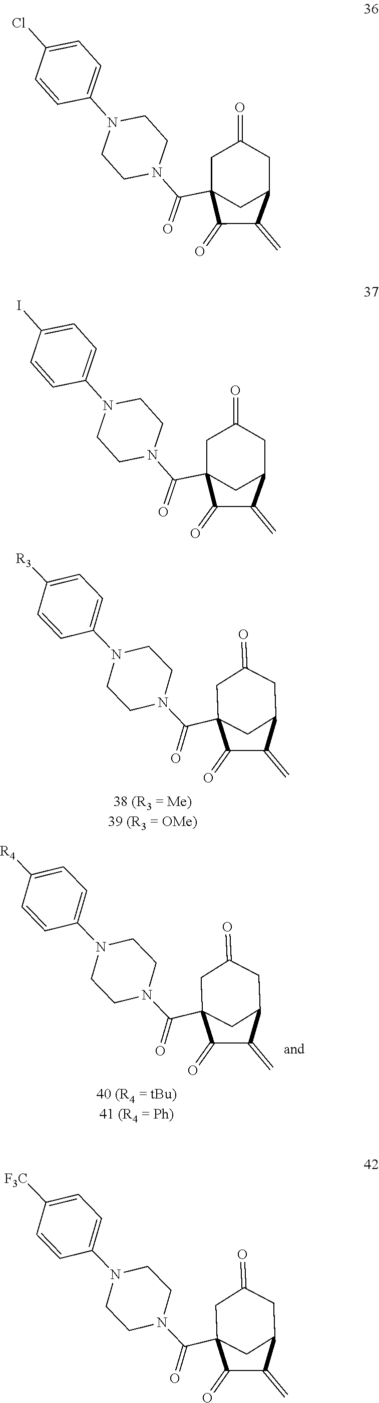

or a pharmaceutically acceptable salt, hydrate, tautomer, or optical isomer thereof.

In some embodiments, the compounds of formula (I) and (IA) comprise bicyclo[3.2.1]octane α-methylene ketone (A) as their pharmacophore and the Michael acceptor α, β-unsaturated carbonyl group as the electrophilic warhead.

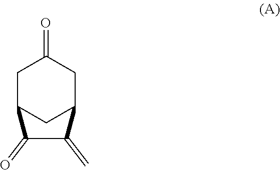

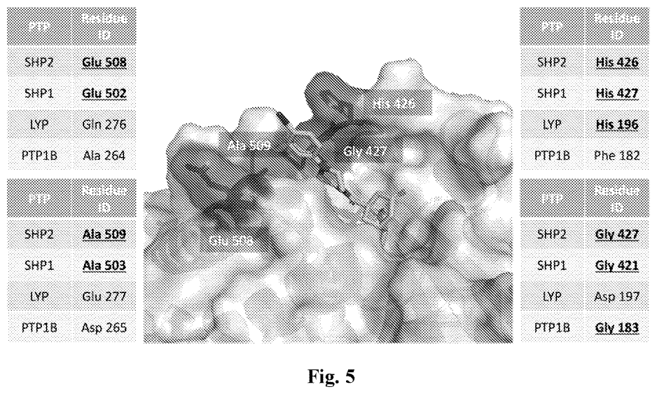

FIG. 5 shows that the removal of the Michael acceptor carbonyl group resulted in a complete loss of SHP2 inhibition efficacy, which indicates that the electrophilic warhead is necessary for inhibition activity.

### Synthesis of Compounds of Formula (I) or (IA):

Compound (7) with a bicyclo[3.2.1]octane core was prepared, as shown in Scheme 1. The compound was synthesized from commercially available 3-methoxybenzoic acid (3). The compound (3) was subjected to a one-pot Birch reduction, alkylation, and hydrolysis to obtain compound (4), which was subsequently converted to methyl ester (5) by reacting with dimethyl sulfate in refluxing acetone. Subsequent radical cyclization followed by ester hydrolysis gave compound (7). Compound (IA) was synthesized from compound (7) in a 3-step reaction sequence, namely EDC-promoted amide formation, allylic C—H oxidation, and Dess-Martin oxidation. Thus, compound (IA) comprising bicyclo[3.2.1]octane α-methylene ketone core and various R₁ and R₂ groups was synthesized.

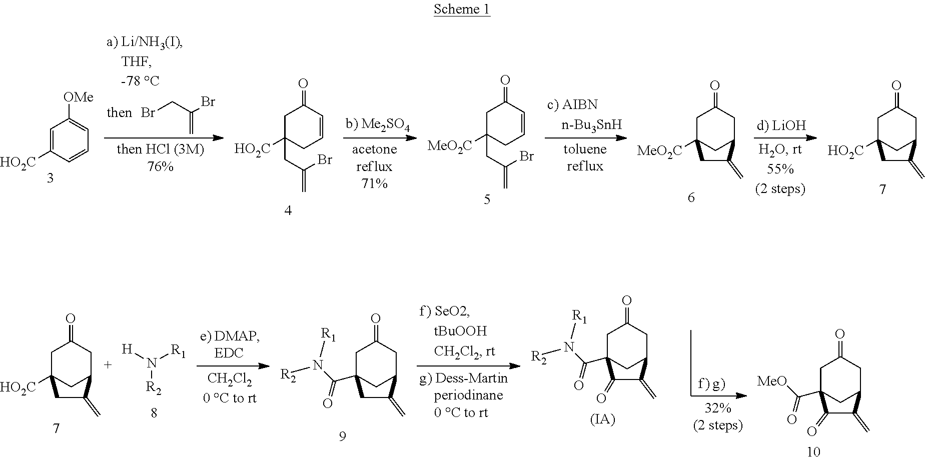

The term “substituted” (e.g., as in “optionally substituted”) refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halo (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups. Non-limiting examples of substituents, which can be bonded to a substituted carbon atom (or other atom, such as nitrogen) include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, alkyl, acyl, cycloalkyl, aryl, aralkyl, heterocyclyl, heteroaryl, or heteroarylalkyl, any alkyl, acyl, cycloalkyl, aryl, aralkyl, heterocyclyl, heteroaryl, or heteroarylalkyl or R can be independently mono- or multi-substituted; or when two R groups bonded to a nitrogen atom or to adjacent nitrogen atoms can, together with the nitrogen atom or atoms to which they are bonded, form a heterocyclyl, the heterocycle can be mono- or independently multi-substituted.

The term “alkyl” refers to substituted or unsubstituted straight-chain and branched alkyl groups and cycloalkyl groups having from 1 to about 20 carbon atoms (e.g., C₁-C₂₀), 1 to 12 carbons (e.g., C₁-C₁₂), 1 to 8 carbon atoms (e.g., C₁-C₈), or, in some embodiments, from 1 to 6 carbon atoms (e.g., C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted or unsubstituted straight-chain and branched divalent alkenyl and cycloalkenyl groups having from 2 to 20 carbon atoms (e.g., C₂-C₂₀), 2 to 12 carbons (e.g., C₂-C₁₂), 2 to 8 carbon atoms (e.g., C₂-C₈) or, in some embodiments, from 2 to 4 carbon atoms (e.g., C₂-C₄) and at least one carbon-carbon double bond. Examples of straight-chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkynyl” refers to an unsaturated monovalent chain of carbon atoms, including at least one triple bond, which may be optionally branched. In various embodiments that include alkynyl, illustrative examples include lower alkynyl, such as C₂-C₆, C₂-C₄ alkynyl, and the like.

The term “hydroxyalkyl” refers to alkyl groups substituted with at least one hydroxyl (—OH) group.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl groups include mono-, bi-, or tri-cyclic alkyl groups. In some embodiments, the cycloalkyl groups can have 3 to about 8-12 ring members, whereas in other embodiments, the number of ring carbon atoms ranges from 3 to 4, 5, 6, or 7. In some embodiments, cycloalkyl groups can have 3 to 6 carbon atoms (e.g., C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “alkoxy” refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryl” refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (e.g., C₆-C₁₄) or from 6 to 10 carbon atoms (e.g., C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “heteroaryl” represents aromatic ring comprising at least one hetero atom such as N, S, O, or Se. Heteroaryl in the present disclosure may be any hetero aryl. Heteroaryl includes, but is not limited to, pyrrolidinyl, azetidinyl, piperidinyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, benzimidazolinyl groups, or any combination thereof.

The terms “halo,” “halogen,” and “halide” group, by themselves or as part of another substituent, mean, unless otherwise stated, a fluorine, chlorine, bromine, or iodine atom. The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl includetrifluoromethyl, 1,1-dichloroethyl, 1,2-dichloroethyl, 1,3-dibromo-3,3-difluoropropyl, perfluorobutyl, —CF(CH₃)₂ and the like.

The term “heterocycloalkyl” refers to a non-aromatic heterocycle where one or more of the ring-forming atoms is/are a heteroatom, such as an O, N, or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion. Also included in the definition of hetero-cycloalkyl are moieties where one or more ring-forming atoms are substituted by 1 or 2 oxo or sulfido groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains O to 3 double bonds. In some embodiments, the heterocycloalkyl group contains 0 to 2 triple bonds.

The term “heterocyclyl” refers to substituted or unsubstituted aromatic and non-aromatic ring compounds containing three or more ring members, of which one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (e.g., C₃-C₈), 3 to 6 carbon atoms (e.g., C₃-C₆) or 6 to 8 carbon atoms (e.g., C₆-C₈).

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The terms “optionally substituted” and “optional substituents” indicate that the groups in question are either unsubstituted or substituted with one or more of the substituents specified. When the groups in question are substituted with more than one substituent, the substituents may be the same or different. When used with the terms “independently”, “independently are,” and “independently selected from,” the groups in question may be the same or different. Certain of the herein defined terms may occur more than once in the structure and, upon such occurrence, each term shall be defined independently of the other.

The term “amine” refers to primary, secondary, and tertiary amines having, e.g., the formula N(group)3 wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The term “compound” as used herein, is meant to include all stereoisomers, geometric isomers, and tautomers of the structures depicted.

The compounds may contain one or more chiral centers, or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, and compositions, methods, uses, and medicaments that include them, may be pure or any of a variety of geometric isomer mixtures. Such mixtures of geometric isomers may include a single configuration at one or more double bonds, while including mixtures of geometry at one or more other double bonds.

The term “solvate” refers to a combination, physical association, and/or solvation of a compound described herein with a solvent molecule such as e.g., a disolvate, monosolvate, or hemisolvate, where the ratio of the solvent molecule to a compound described is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates.

Solvated forms of the compounds can be prepared with a pharmaceutically acceptable solvent. Examples of the solvents include, but are not limited to, water, methanol, and ethanol, and it is intended that the disclosure includes both solvated and unsolvated forms of above-described compounds. One type of solvate is a hydrate. A “hydrate” relates to a particular subgroup of solvates where the solvent molecule is water. Solvates typically can function as pharmacological equivalents. The preparation of solvates is known in the art. For example, M. Caira et al., J. Pharmaceut. Sci., 2004, 93(3): 601-611, describes the preparation of solvates of fluconazole with ethyl acetate and with water. Similar preparation of solvates, hemisolvates, hydrates, and the like are described by E. C. van Tonder et al., AAPS Pharm. Sci. Tech., 2004, 5(1): Article 12, and A. L. Bingham et al, 200, Chem. Commun., 603-604. A typical, non-limiting, process of preparing a solvate would involve dissolving a compound in a desired solvent (organic, water, or a mixture thereof) at temperatures above 20° C. to about 25° C., then cooling the solution at a rate sufficient to form crystals, and isolating the crystals by known methods, e.g., filtration. Analytical techniques, such as infrared spectroscopy, can be used to confirm the presence of the solvate in a crystal of the solvate.

The term “pharmaceutically acceptable salt” refers to salts or zwitterionic forms of compounds described herein.

Examples of the salts of the compounds described herein include, but are not limited to, hydrochloride salt, hydrobromide salt, hydroiodide salt, sulfate salt, bisulfate salt, 2-hydroxyethansulfonate salt, phosphate salt, hydrogen phosphate salt, acetate salt, adipate salt, alginate salt, aspartate salt, benzoate salt, bisulfate salt, butyrate salt, camphorate salt, camphorsulfonate salt, digluconate salt, glycerolphosphate salt, hemisulfate salt, heptanoate salt, hexanoate salt, formate salt, succinate salt, fumarate salt, maleate salt, ascorbate salt, isethionate salt, salicylate salt, methanesulfonate salt, mesitylenesulfonate salt, naphthylenesulfonate salt, nicotinate salt, 2-naphthalenesulfonate salt, oxalate salt, pamoate salt, pectinate salt, persulfate salt, 3-phenylproprionate salt, picrate salt, pivalate salt, propionate salt, trichloroacetate salt, trifluoroacetate salt, phosphate salt, glutamate salt, bicarbonate salt, paratoluenesulfonate salt, undecanoate salt, lactate salt, citrate salt, tartrate salt, gluconate salt, methanesulfonate salt, ethanedisulfonate salt, benzene sulfonate salt, and p-toluenesulfonate salt.

Any reference compounds of the present disclosure appearing herein are intended to include compounds of the present disclosure as well as pharmaceutically acceptable salts or hydrates thereof.

Further provided is a pharmaceutical composition comprising a compound of formula (I) or (IA) or a pharmaceutically acceptable salt, hydrate, tautomer, or optical isomer of either of the foregoing and a pharmaceutically acceptable carrier, excipient, or diluent.

Any suitable route can be used to administer the compound of formula (I) or (IA). In some embodiments, the compound of formula (I) or (IA) can be administered orally, parenterally, subcutaneously, intramuscularly, intravenously, intradermally, and rectally.

In some embodiments, a pharmaceutical composition further comprises at least one additional pharmaceutically active agent. Pharmaceutical composition can be prepared by combining one or more compounds of formula (I) or (IA) or a pharmaceutically acceptable salt, solvate, tautomer, or stereoisomer of either with a pharmaceutically acceptable carrier or excipient or diluent and, optionally, one or more additional pharmaceutically active agents.

Further provided is a method of treating or inhibiting cancer comprising administering to a patient a therapeutically effective amount of a compound of formula (I) or (IA) or a pharmaceutically acceptable salt, hydrate, tautomer, or optical isomer of either optionally as a pharmaceutical composition comprising the compound and a pharmaceutically acceptable carrier, excipient, or diluent. The method involves covalent inhibition of protein tyrosine phosphatase SHP2.

In some embodiments, the cancer is associated with SHP2. Examples of cancers include, but are not limited to, leukemia, lung, breast, gastric, laryngeal, liver, and oral.

Further provided is a method of using the compounds of formula (I) or (IA) for treating diseases which are associated with SHP2.

Further provided is a method of inhibiting SHP2 comprising administering to a patient a therapeutically effective amount of a compound of formula (I) or (IA) or a pharmaceutically acceptable salt, hydrate, tautomer, or optical isomer of either optionally as a pharmaceutical composition comprising the compound and a pharmaceutically acceptable carrier, excipient, or diluent.

The compounds of the present disclosure can also be used in combination with other compounds or known drugs that are used to treat cancer, including natural product or natural product-derived covalent drugs. Examples of the known natural products or natural product-derived covalent drugs that can be used to treat cancer include, but are not limited to, aspirin, penicillin, finasteride, orlistat, fumaderm, triptolide, minnelide, sonolisib (PX-866), E6201, dimethylamino parthenolide, bardoxolone methyl, ludongnin, eriocalyxin B, jungermannenone C, enmein, adenanthin, and oridonin.

Provided is a pharmaceutical combination for treating cancer in a patient in need thereof. The pharmaceutical combination comprises (i) a compound of formula (I) or (IA) or a pharmaceutically acceptable salt, hydrate, tautomer, or optical isomer of either, (ii) an additional therapeutic agent, and (iii) optionally at least one pharmaceutically acceptable carrier, excipient, or diluent.

The term “pharmaceutical combination” refers to a pharmaceutical therapy resulting from the mixing or combining of more than one active ingredient. For a combination, the compound of formula (I) or (IA) or a pharmaceutically acceptable salt, hydrate, tautomer, or optical isomer of either and at least one additional therapeutic agent can be administered to a patient simultaneously or sequentially by the same or different route of administration in a single composition or as two separate compositions to achieve the desired effect. The therapeutic agent can be administered in an amount to provide its desired therapeutic effect. The effective dosage range for each therapeutic agent is well-known in the art, and the therapeutic agent is administered to a patient in need thereof within such established ranges.

The terms “treat,” “treating,” “treated,” and “treatment” (with respect to a disease or condition) are used to describe an approach for obtaining beneficial or desired results, preferably clinical results, and include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

The term “pharmaceutical composition” includes a therapeutically effective amount of one or more compounds for treating a patient, such as a patient with cancer. The composition may include other components and/or ingredients, including, but not limited to, other therapeutically active compounds and/or one or more pharmaceutically acceptable carriers, diluents, excipients, and the like. The pharmaceutical composition can be prepared using known conventional methods, as described in pertinent textbooks, e.g., Remington: The Science and Practice of Pharmacy, 23^{rd} Ed.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10)glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffered solutions; and (21) other non-toxic compatible substances employed in pharmaceutical formulations.

The term “therapeutic effect” refers to a beneficial local or systemic effect in animals, particularly mammals and, more particularly, humans, caused by the administration of a compound or a pharmaceutical composition or combination comprising same.

The term “therapeutically effective amount” means the amount of a compound that is effective to treat a disease or a disorder, such as a SHP2-driven cancer, at a reasonable benefit/risk ratio. The therapeutically effective amount of such compound will vary depending upon the patient and the disease or disorder being treated, the weight and age of the patient, the severity of the disease or disorder, the manner of administration, and the like, which can readily be determined by one of skill in the art.

The compounds can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the patient including, but not limited to, by oral, intravenous, intratumoral, intramuscular, subcutaneous, transdermal, topical, and like routes of administration.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

Useful dosages of the compounds can be determined by comparing their in vitro activity with their in vivo activity in animal models. Methods of the extrapolation of effective dosages in mice and other animals to human subjects are known in the art. Indeed, the dosage of the compounds can vary significantly depending on the condition of the subject, the age of the subject, the type of disease the subject is experiencing or at risk of experiencing, the particular compounds used, how advanced the pathology is, the route of administration of the compounds and the possibility of co-usage of other therapeutic treatments or additional drugs in combination therapies. The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The compositions comprising the compound (s) can be formulated in a unit dosage form, each dosage containing from about 5 to about 1,000 mg (1 g), more usually about 100 mg to about 500 mg, of the compound, i.e., active ingredient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 ng to about 500 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 ng to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compound can be administered in an amount ranging from about 1 mg/kg to about 100 mg/kg. In some embodiments, the compound can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

The term “patient” includes human and non-human animals such as companion animals (dogs and cats and the like) and livestock animals. Livestock animals are animals raised for food production. The patient to be treated is preferably a mammal, in particular a human being.

Covalent inhibitors work by forming a covalent bond with a reactive functional group in the target enzyme. Consequently, the kinetics for enzyme inactivation by a covalent inhibitor is not an equilibrium process as with reversible inhibition. The potency of a reversible inhibitor can be measured by its IC₅₀ value. Unlike reversible inhibitors, where inhibition is only concentration-dependent, covalent inhibitors show both time- and concentration-dependent target inhibition (a covalent bond formation is a time-dependent event). If sufficient time is available, covalent inhibitors can completely neutralize the target, rather than go to equilibrium. As a result, the potency and selectivity of a covalent inhibitor cannot be determined by IC₅₀ values; instead, the rate of inactivation of the target (k_{inact}) and the inhibition constant (K_{I}) were determined. The bimolecular rate constant (k_{inact}/K_{I}), was chosen to rank the potency of covalent inhibitors (Ruddraraju et al., 2017, Molecular Biosystems 13(7), 1257-1279). Table 1 shows the results of k_{inact}/K_{I} of the compounds of formula (I) and (IA).

### EXAMPLES

The following examples serve to illustrate the present disclosure. The examples are not intended to limit the scope of the claimed invention in any way.

### Biological Evaluations:

PTPs catalyze the dephosphorylation of tyrosine-phosphorylated protein substrates through a conserved mechanism that relies on a catalytic cysteine residue (Zhang, Z.-Y et al., 2003, Prog. Nucleic Acid Research Molecular Biology, 73, 171-220). This provides a mechanistic rationale for the assessment of electrophilic Michael acceptors, such as those synthesized in the present disclosure as potential covalent inhibitors of the SHP2. The enzymatic cleavage of para-nitrophenyl phosphate by SHP2 was measured over time in the presence of 100 μM inhibitor or DMSO, to determine the observed first-order rate constant (k_{obs}). Based on k_{obs} values, compounds were further evaluated in a dose-dependent manner to determine the second-order rate constant (k_{inact}/K_{I}) indicating the overall efficacy of the compounds, which was used to assess the structure-activity relationship (SAR) for the analogs of interest and design additional analogs (see Table 1). The representative core electrophile, compound (10) (see Entry 2, Table 1), exhibited a k_{inact}/K_{I} value of 1,228.8 M⁻¹Min⁻¹ for SHP2, displaying an approximately 6-fold increase in efficacy compared to the reported covalent PTP inhibitor phenyl vinyl sulfonate (PVSN) (k_{inact}/K_{I}=220.74 M⁻¹Min⁻¹) (Liu, S., et al., J. Am. Chem. Soc. 2008, 130, 8251-8260). It has been known that modifications to the electronegativity of substituents proximal to electrophilic centers alter the reactivity of the electrophile. The n-butyl amide substitution of compound (11) has a k_{inact}/K_{I} value of 856.8 M⁻¹Min⁻¹, slightly less potent than compound (10), likely resulting from the decreased electronegativity of the carboxamide relative to the ester. The compound (12) having N,N-diethylamide analog is more effective than compound (11) and the cyclic matched molecular pair (MMP) compound (17). Increasing the ring size to a 6- or 7-membered ring did increase the efficacy of the analogs, suggesting a preference for hydrophobic bulk at this position.

TABLE 1
In vitro efficacy of the first-generation compounds against SHP2

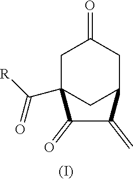

| Entry | Compound | R-Group | k_{inact}/K_{I} (M⁻¹Min⁻¹) | 95% C.I. (M⁻¹Min⁻¹) |
|---|---|---|---|---|
| 1 | PVSN | 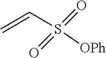 | 210.96 | 186.78 to 235.14 |
| 2 | 10 | OMe | 1,228.8 | 1,181.4 to 1,276.2 |
| 3 | 11 | 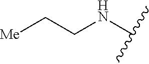 [SMILES: *NCCC] | 865.8 | 830.4 to 883.2 |
| 4 | 12 | 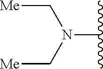 [SMILES: *N(CC)CC] | 1,051.2 | 1,010.4 to 1,091.4 |
| 5 | 17 | 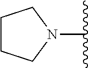 [SMILES: *N1CCCC1] | 803.4 | 723.6 to 883.2 |
| 6 | 19 | 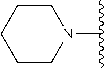 [SMILES: *N1CCCCC1] | 1,162.2 | 1,114.2 to 1,210.2 |
| 7 | 20 | 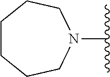 [SMILES: *N1CCCCCC1] | 1,361.4 | 1,324.8 to 1,397.4 |
| 8 | 21 | 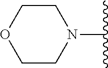 [SMILES: *N1CCOCC1] | 958.8 | 921.0 to 996.6 |
| 9 | 22 | 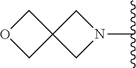 [SMILES: *N1CC2(COC2)C1] | 774.0 | 744.6 to 803.4 |
| 10 | 23 | 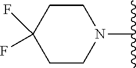 [SMILES: *N1CCC(F)(F)CC1] | 2,026.8 | 1,987.2 to 2,065.8 |
| 11 | 25 | 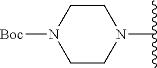 [SMILES: *N1CCN(C(=O)OC(C)(C)C)CC1] | 1,687.2 | 1,600.8 to 1,773.6 |
| 12 | 26 | 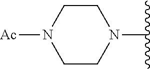 [SMILES: *N1CCN(C(C)=O)CC1] | 923.4 | 868.8 to 978.6 |
| 13 | 30 | 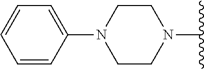 [SMILES: *N1CCN(c2ccccc2)CC1] | 2,377.2 | 2,377.2 to 2,502.0 |
| 14 | 33 | 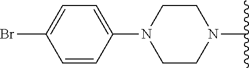 [SMILES: *N1CCN(c2ccc(Br)cc2)CC1] | 22,006.4 | 19,881.1 to 24,328.9 |

Modifications to the piperidine scaffold were then explored. Corroborating the above observation for the preference of hydrophobic substituents at this position, the morpholine analog compound (21) and a spirocyclic morpholine isostere compound (22) displayed an approximate 2-fold decrease in efficacy. 4,4-Difluoropiperidine-bearing analog compound (23), in comparison, was a more effective SHP2 inhibitor with a k_{inact}/K_{I} value of 2,206.8M⁻¹Min⁻¹. Both the N-Boc piperidine and N-acetyl piperazine analogs compounds (25 and 26) were able to inhibit the catalytic activity of SHP2, providing a synthetically accessible piperazine core that would be amenable to further SAR. Phenyl-substituted analog compound (30) resulted in a small improvement in efficacy. The addition of a bromine atom at the para position of the phenyl group (compound (32)) led to a 9.25-fold increase in efficacy relatively to the unsubstituted phenyl ring, with a k_{inact}/K_{I} value of 22,006.4 M⁻¹Min⁻¹, providing an approximately 19-fold improvement in efficacy over the unsubstituted piperidine analog (19). Because of this substantial increase in efficacy, analogs with further modifications at this position were synthesized and investigated (See Scheme 1, compounds 35-42, and Table 2).

TABLE 2
In vitro efficacy of the second-generation compounds against SHP2

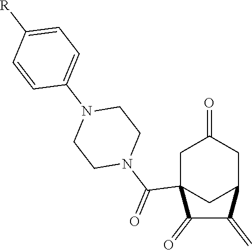

| | Compound | kinact/K_{I} | 95% C.I. | K_{inact} | K_{I} |
|---|---|---|---|---|---|
| Entry | R is | (M⁻¹Min⁻¹) | (M⁻¹Min⁻¹) | (Sec⁻¹) | (μM) |
| 1 | H (30) | 2,377.2 | 2,377.2 to 2,502 | N/A | N/A |
| 2 | F (35) | 5,855.6 | 5,274.4 to 6,500 | 1.095 × 10⁻² | 112.2 |
| 3 | Cl (36) | 13,194 | 12,828 to 13,560 | N/A | N/A |
| 4 | Br (33) | 22,006.4 | 19,881.1 to 24,329 | 2.19 × 10⁻² | 69.71 |
| 5 | I (37) | 23,754.0 | 22,356 to 25,158 | N/A | N/A |
| 6 | CH₃ (38) | 6,067.4 | 5,242.2 to 6,526 | 1.718 × 10⁻² | 176.2 |
| 7 | OMe (39) | 2,788.2 | 2,659.8 to 2,916 | N/A | N/A |
| 8 | tBu (40) | 17,442.0 | 16,674.0 to 18,204 | N/A | N/A |
| 9 | Ph (41) | 18,634.1 | 16,249.6 to 21,400 | 3.174 × 10⁻³ | 10.22 |
| 10 | CF₃ (42) | 25,013.6 | 22,054.2 to 28,340 | 1.994 × 10⁻² | 47.83 |

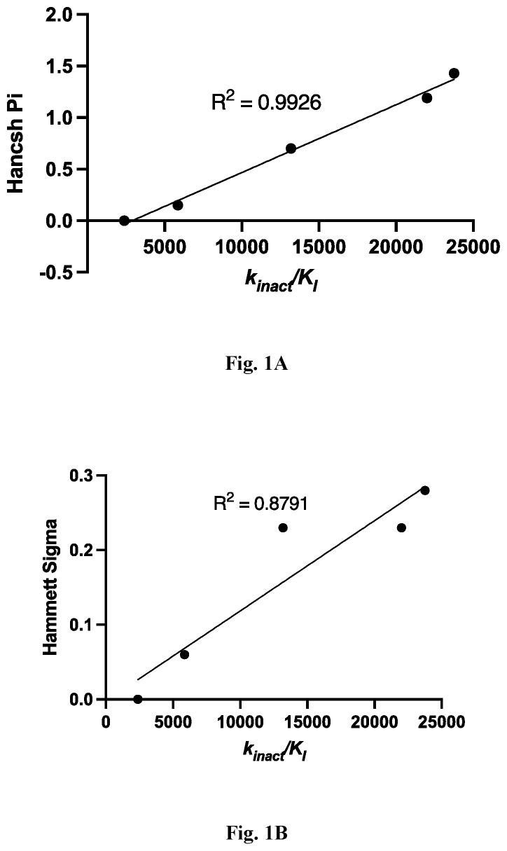

To complete the halogen series, the effect of fluorine, chlorine, and iodine were explored at the para-position (Table 2). The trend observed was H<F<Cl<Br≈I, indicated that the additional hydrophobicity of the halogens contributes favorably to the molecules binding. Plotting the k_{inact}/K_{I} values for these analogs against their corresponding Hansch pi and sigma values revealed a clear correlation between hydrophobicity and efficacy (FIG. 1, A) as well as for electronegativity and efficacy, although to a lesser extent (FIG. 1 B). Additional modifications were explored here to further probe the tolerance of functional groups of varying hydrophobicity. Replacement of the bromide with a methyl or methoxy group resulted in a significant loss in activity. Replacing the bromide with a tert-butyl or phenyl moiety rescued the activity, again supporting the observation that hydrophobic substituents were preferred at this position. Modification of the N-phenyl ring with a trifluoromethyl group at the para position resulted in the most effective analog with a k_{inact}/K_{I} value of 25,013.6 M⁻¹Min⁻¹.

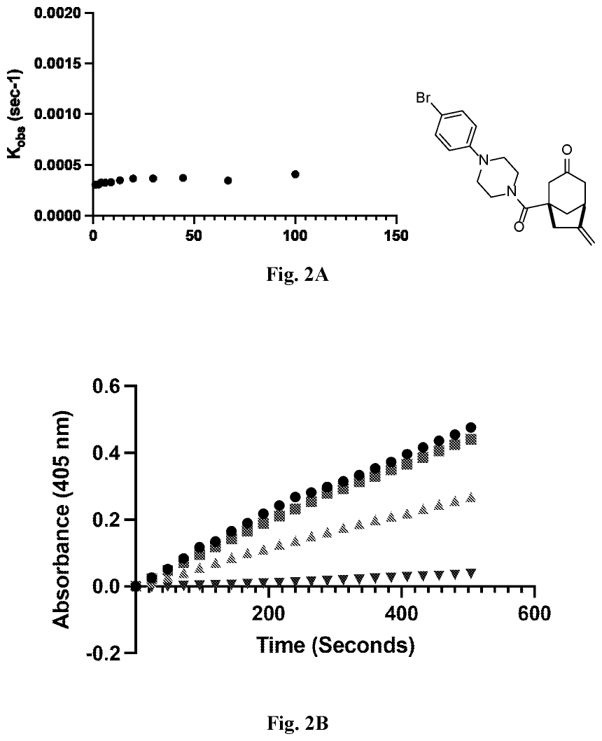

Compound (34) was synthesized to evaluate the effects of removing the electrophile warhead on compound efficacy. Removal of the Michael acceptor carbonyl resulted in a complete loss of SHP2 inhibition efficacy (FIG. 2), suggested that the electrophilic warhead is required for inhibition. Thus, SAR study supports the requirement for the presence of the electrophilic warhead. Additionally, an observed trend for increasing efficacy was observed with increasing ring size adjacent to the bicyclic warhead. Finally, there was a clear preference for hydrophobic, electronegative substituents at the para position of the terminal phenyl ring. All together, these modifications resulted in compound (42), displaying a 29-fold increase in efficacy over 11 and 119-fold increase over PVSN.

### Characterization of Active Compounds:

The efficacy of covalent inhibitors can be broken down into the initial reversible binding of the compounds (K_{I}), and the rate of covalent bond formation and enzyme inactivation (k_{inact}). These two constants can be used individually, or as a ratio to determine the overall efficacy of the inhibitor's efficacy. To investigate the contribution to reversible binding compound (34) was evaluated, which lacks the carbonyl of the Michael acceptor to prevent the formation of a covalent adduct. This analog was inactive against SHP2, suggesting that this moiety is required for the inhibition and inactivation of SHP2, supporting the proposed covalent mechanism of action for this compound series.

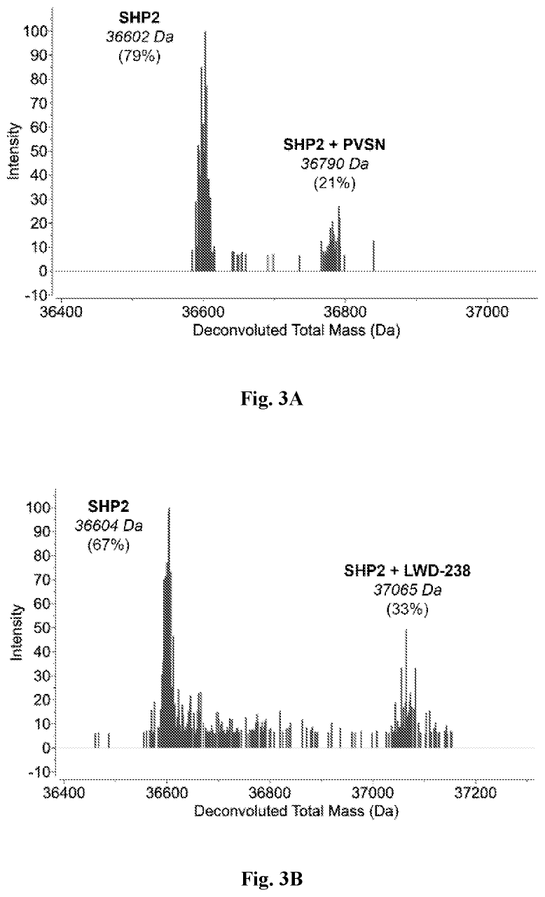

To further support a covalent mechanism of action and engagement of the SHP2 active site, a jump-dilution assay was employed both in the presence and absence of the known reversible PTP active site competitive inhibitor vanadate (VO4) (Seiner, D. R. et al., Chem. Res. Toxicol. 2007, 20, 1315-1320) In this experiment, SHP2 was co-incubated with 100 μM compound (10) and 500 μM vanadate or DMSO for 10 minutes. The enzyme-inhibitor complex was then rapidly diluted 100-fold into a buffered solution containing 10 mM pNPP, and the absorbance at 405 nm was immediately monitored over time to determine the remaining enzymatic activity. As expected, the SHP2·VO4³⁻ complex rapidly dissociated upon dilution, and SHP2 fully recovered activity compared to the DMSO-treated control (FIG. 3). In comparison, the SHP2·(10) complex displayed negligible residual enzymatic activity, suggesting that compound (10) irreversibly inhibited SHP2. When SHP2 was co-incubated with both (10) and VO4³⁻, the enzyme-inhibitor complex did show substantial residual activity, suggesting that compound (10) interacts with the active site of SHP2, likely by forming a covalent bond with the active site cysteine.

### Covalent Adduction Formation by LC-MS:

To validate covalent modification of SHP2 by the active compounds, adduct formation on SHP2 was measured by intact protein liquid chromatography-mass spectrometry (LC-MS). With intact protein LC-MS, multiply charged states of the protein species can be deconvoluted to achieve the total mass(es). Compounds were incubated with 10 μM SHP2 for 60 minutes before being quenched with acetonitrile for LC-MS analysis by electrospray ionization (ESI) in positive mode. When 10 μM SHP2 was treated with 20 μM PVSN, a major mass shift of 188 Da was observed with an overall 21% adduct formation on SHP2 by single quadrupole LC-MS. Compound (37) incubated with SHP2 resulted in a mass shift of 461 Da, corresponding to the SHP2-37 adduct, with an overall adduct formation of 33%. Similar SHP2 adduct formation was also observed with compounds (10). (33), and (42) (FIGS. 3A and 3B).

### Selectivity of Compound (37) for SHP2:

During active compound characterization, other common PTPs, including SHP1. PTP1B, and LYP, were counter-screened. Compounds were incubated for 10 minutes with the corresponding PTP (4 μM) before being diluted 50-fold into a solution containing excess substrate para-nitrophenyl phosphate (10 mM) to monitor the remaining activity. One of the analogs compound (37), displayed an interesting selectivity profile. The IC₅₀ value for SHP2 after 10 minutes of preincubation with compound (37) was 15.42 μM (Table 3). Strikingly, its IC₅₀ value for SHP1 after the same length of preincubation was 212.0 μM, representing a 13.75-fold window of selectivity. This was not expected, given their high sequence and structural homology. Compound (37) was even more selective against LYP (IC₅₀=495 μM) and PTP1B (IC₅₀=677.3 μM). While unexpected, the lower sequence homology provides a rationale for the observed selectivity.

TABLE 3
shows IC50 values after 10 minutes of preincubation.

| | | | Fold- Selective |
|---|---|---|---|
| ID | PTP | 10 Minute IC₅₀ (μM) | (10 Minutes) |
| 37 | SHP2 | 15.42 | — |
| | SHP1 | 212.0* | 13.75 |
| | LYP | 495.3* | 31.12 |
| | PTP1B | 677.3* | 44.56 |

*Curve was not completed due to solubility limitations; thus, values are extrapolated.

### General Information

i)) NMR spectra were recorded on Bruker spectrometers (¹H at 400 MHz, 500 MHz, 800 MHz and ¹³C at 100 MHz, 125 MHz, 200 MHz). Chemical shifts (δ) were given in ppm with reference to solvent signals [¹H NMR: CHCl₃ (7.26); ¹³C NMR: CDCl₃ (77.16)].
ii) Column chromatography was performed on silica gel.
iii) All reactions sensitive to air or moisture were conducted under argon atmosphere in dry and freshly distilled solvents under anhydrous conditions, unless otherwise noted. Anhydrous THF and toluene were distilled over sodium benzophenone ketyl under argon. Anhydrous CH₂Cl₂ was distilled over calcium hydride under argon. All other solvents and reagents were used as obtained from commercial sources without further purification.
iv) HPLC purity data was performed on Agilent 1260 Infinity Quaternary LC. Column: Kinetex 2.6 μm C18 100 Å, LC Column 30×2.1 mm. Method: Gradient eluent from H₂O (added 0.1% formic acid): MeCN (added 0.1% formic acid)=95:5 to 5:95, in 4 min.

### EXAMPLES

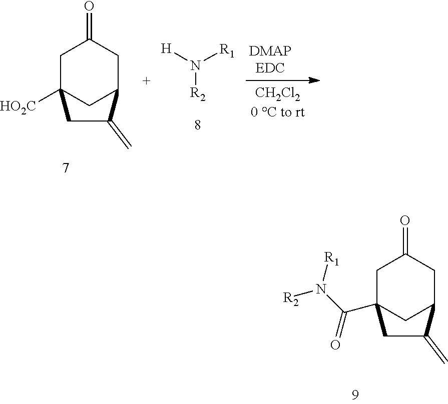

Compound (7) was prepared according to the procedure well-known in the art (Tetrahedron: Asymmetry 1991, 2, 875-878; Org. Lett. 2011, 13, 5286-5289; Angew. Chem. Int. Ed. 2019, 58, 15731-15735), which are hereby specifically incorporated by reference for their teachings regarding the same.

### General Procedure for the Synthesis of Compound (9)

To a solution of carboxylic acid (7) (1.0 equiv) in dry DCM (0.03 M) at 0° C., EDCI (1.2 equiv) was added. After stirring at room temperature for 15 min, DMAP (0.1 equiv) and amine (8) (1.1 equiv) dissolved in small amount of DCM were added to the mixture at 0° C. The resulting mixture was warmed to room temperature and kept stirring at that temperature for 12 h. The reaction was quenched with aqueous HCl (1 N). The phases were separated, and the aqueous layer was extracted with DCM. The combined organic phases were dried over Na₂SO₄, filtered, and the solvent was removed in vacuo. The residue was purified by silica gel flash chromatography to afford amide (9).

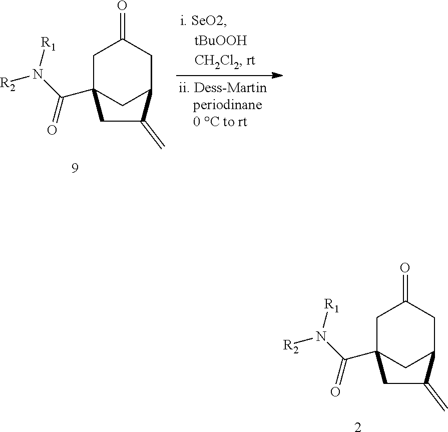

### General Procedure for the Synthesis of Compound (2)

To a stirred solution of compound (9) (1.0 equiv) in DCM (0.02 M), SeO₂ (0.5 equiv) was added in one portion, and a solution of tBuOOH (6.5 equiv, 70% in H₂O,) was added dropwise. The solution was vigorously stirred at room temperature for 6 h, and then the reaction was diluted with water. The mixture was extracted with DCM, and the combined organic layers were washed with sat. Na₂S₂O₃, brine, dried over Na₂SO₄, filtered, and concentrated in vacuo. The residue was purified by silica gel flash chromatography to give allylic alcohol. A stirred solution of the allylic alcohol (1.0 equiv) in DCM (0.02 M) was cooled to 0° C., then DMP (1.5 equiv) was added in several portions. The reaction was warmed to room temperature and stirred for 4 h before diluted with water. The mixture was extracted with DCM, and the combined organic layers were washed with brine, dried over Na₂SO₄, filtered, and concentrated in vacuo. The residue was purified by silica gel flash chromatography to provide enone (2). For each compound, the yield was calculated for 3 steps.

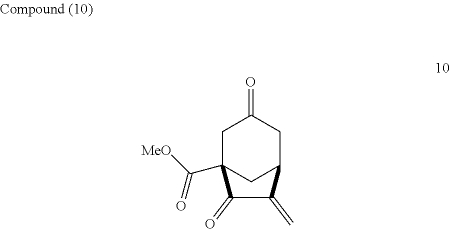

Compound 10, colorless oil, yield: 32%.

¹H NMR (400 MHz, CDCl₃) δ 6.17 (d, J=0.5 Hz, 1H), 5.57 (d, J=0.5 Hz, 1H), 3.79 (s, 3H), 3.48-3.37 (m, 1H), 2.90 (d, J=17.8 Hz, 1H), 2.79 (d, J=17.8 Hz, 1H), 2.76-2.73 (m, 1H), 2.72-2.65 (m, 1H), 2.58-2.52 (m, 1H), 2.20 (d, J=12.3 Hz, 1H);

¹³C NMR (125 MHz, CDCl₃) δ 205.8, 199.6, 170.4, 145.8, 121.6, 58.2, 53.0, 48.9, 47.9, 36.7, 36.1;

¹⁹F NMR (470 MHz, CDCl₃) δ −62.67;

HRMS (ESI): m/z Calc. for C₁₁H₁₃O₄ [M+H]⁺: 209.0808. found: 209.0809.

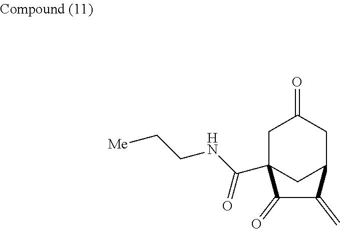 [SMILES: C=C1C(=O)[C@@]2(C(=O)NCCC)CC(=O)C[C@@H]1C2]

Compound 11, colorless oil, yield: 47%.

¹H NMR (500 MHz, CDCl₃) δ 7.52 (br, 1H), 6.18 (s, 1H), 5.58 (s, 1H), 3.43-3.33 (m, 1H), 3.25 (ddt, J=7.1, 5.7, 3.6 Hz, 2H), 2.97 (d, J=16.7 Hz, 1H), 2.73 (dd, J=16.7, 3.9 Hz, 1H), 2.64-2.43 (m, 3H), 2.39-2.32 (m, 1H), 1.65-1.50 (m, 2H), 0.93 (t, J=7.4 Hz, 3H);

¹³C NMR (125 MHz, CDCl₃) δ 205.8, 204.1, 169.1, 147.4, 121.9, 56.6, 51.4, 48.8, 41.1, 36.4, 35.0, 22.7, 11.5;

HRMS (ESI): m/z Calc. for C₁₃H₁₈NO₃ [M+H]⁺: 236.1281. found: 236.1282.

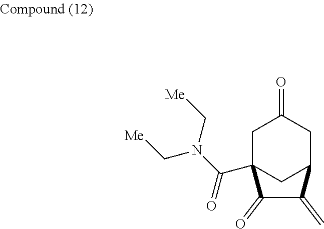 [SMILES: C=C1C(=O)[C@@]2(C(=O)N(CC)CC)CC(=O)C[C@@H]1C2]

Compound 12, colorless oil, yield: 58%.

¹H NMR (500 MHz, CDCl₃) δ 6.15 (s, 1H), 5.57 (s, 1H), 3.43 (s, 2H), 3.33 (s, 1H), 3.15 (d, J=17.7 Hz, 1H), 3.00 (s, 2H), 2.81-2.61 (m, 2H), 2.51 (d, J=16.8 Hz, 2H), 2.21 (d, J=12.9 Hz, 1H), 1.25-0.94 (m, 6H);

¹³C NMR (125 MHz, CDCl₃) δ 206.8, 200.7, 168.1, 146.7, 121.2, 58.3, 50.9, 49.4, 42.4, 40.6, 37.6, 36.6, 13.0, 12.7;

HRMS (ESI): m/z Calc. for C₁₄H₂₀NO₃ [M+H]⁺: 250.1438. found: 250.1440.

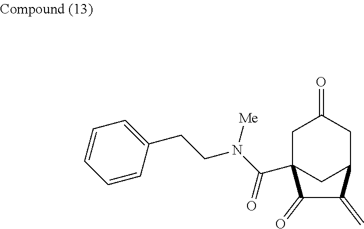 [SMILES: C=C1C(=O)[C@@]2(C(=O)N(C)CCc3ccccc3)CC(=O)C[C@@H]1C2]

Compound 13, white solid, yield: 43%.

¹H NMR (500 MHz, CDCl₃) δ 7.29-7.12 (m, 5H), 6.17 (s, 1H), 5.57 (s, 1H), 3.68-3.02 (m, 5H), 2.92-2.86 (m, 2H), 2.78-2.52 (m, 6H), 2.17 (d, J=12.6 Hz, 1H);

¹³C NMR (125 MHz, CDCl₃) δ 206.6, 200.9, 168.5, 146.5, 139.1, 129.0, 128.7, 126.5, 121.5, 58.3, 51.6, 50.7, 49.4, 37.6, 37.0, 36.7, 33.4;

HRMS (ESI): m/z Calc. for C₁₉H₂₂NO₃ [M+H]⁺: 312.1594. found: 312.1596.

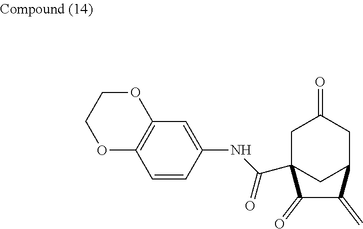 [SMILES: C=C1C(=O)[C@@]2(C(=O)Nc3ccc4c(c3)OCCO4)CC(=O)C[C@@H]1C2]

Compound 14, pale yellow oil, yield: 50%.

¹H NMR (500 MHz, CDCl₃) δ 9.31 (s, 1H), 7.22 (s, 1H), 6.96 (dd, J=8.7, 2.5 Hz. 1H), 6.80 (d, J=8.7 Hz, 1H), 6.24 (d, J=1.2 Hz, 1H), 5.64 (d, J=1.0 Hz, 1H), 4.23 (m, 4H), 3.38 (d, J=4.1 Hz, 1H), 3.02 (d, J=17.0 Hz, 1H), 2.75 (dd, J=16.8, 4.0 Hz, 1H), 2.68 (d, J=17.0 Hz, 1H), 2.61-2.52 (m, 2H), 2.49-2.39 (m, 1H);

¹³C NMR (125 MHz, CDCl₃) δ 205.3, 204.1, 166.8, 147.2, 143.6, 140.8, 131.1, 122.5, 117.3, 113.8, 110.0, 64.5, 64.4, 57.0, 51.3, 48.8, 36.3, 34.9;

HRMS (ESI): m/z Calc. for C₁₈H₁₈NO₅ [M+H]⁺: 328.1180. found: 328.1182.

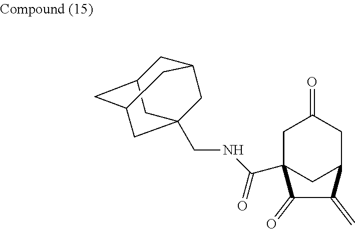 [SMILES: C=C1C(=O)[C@@]2(C(=O)NCC34CC5CC(CC(C5)C3)C4)CC(=O)C[C@@H]1C2]

Compound 15, white solid, yield: 38%.

¹H NMR (500 MHz, CDCl₃) δ 7.60 (s, 1H), 6.20 (s, 1H), 5.60 (s, 1H), 3.35 (s, 1H), 3.06 (dd, J=13.3, 6.4 Hz, 1H), 2.99 (d, J=17.0 Hz, 1H), 2.93 (td, J=14.1, 13.3, 6.0 Hz, 1H), 2.73 (dd, J=16.7, 3.7 Hz, 1H), 2.61 (d, J=17.0 Hz, 1H), 2.55 (d, J=17.3 Hz, 1H), 2.50 (d, J=12.7 Hz, 1H), 2.40-2.33 (m, 1H), 1.98 (s, 3H), 1.71 (d, J=12.1 Hz, 3H), 1.63 (d, J=11.4 Hz, 3H), 1.50 (s, 6H);

¹³C NMR (125 MHz, CDCl₃) δ 205.9, 204.2, 169.3, 147.4, 122.0, 56.8, 51.7, 50.9, 48.9, 40.4, 37.0, 36.5, 35.0, 33.8, 28.3;

HRMS (ESI): m/z Calc. for C₂₁H₂₈NO₃ [M+H]⁺: 342.2064. found: 342.2066.

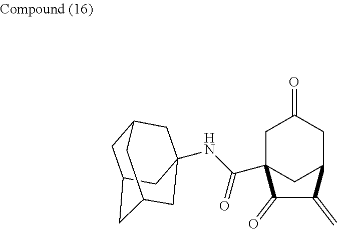 [SMILES: C=C1C(=O)[C@@]2(C(=O)NC34CC5CC(CC(C5)C3)C4)CC(=O)C[C@@H]1C2]

Compound 16, white solid, yield: 26%.

¹H NMR (500 MHz, CDCl₃) δ 7.23 (s, 1H), 6.15 (d, J=1.1 Hz, 1H), 5.55 (s, 1H), 3.31 (d, J=4.2 Hz, 1H), 2.93 (d, J=16.9 Hz, 1H), 2.70 (dd, J=16.7, 4.0 Hz, 1H), 2.53 (ddd, J=22.5, 16.7, 2.5 Hz, 2H), 2.41 (d, J=12.7 Hz, 1H), 2.33 (ddt, J=10.2, 5.3, 2.6 Hz, 1H), 2.13-2.05 (m, 3H), 2.00 (s, 6H), 1.67 (s, 6H);

¹³C NMR (125 MHz, CDCl₃) δ 205.9, 204.0, 167.8, 147.5, 121.5, 57.0, 51.9, 51.6, 48.7, 41.5, 36.3, 36.2, 34.8, 29.4;

HRMS (ESI): m/z Calc. for C₂₀H₂₆NO₃ [M+H]⁺: 328.1907. found: 328.1906.

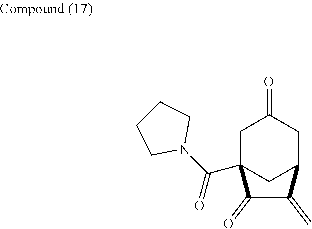 [SMILES: C=C1C(=O)[C@@]2(C(=O)N3CCCC3)CC(=O)C[C@@H]1C2]

Compound 17, white solid, yield: 29%.

¹H NMR (500 MHz, CDCl₃) δ 6.15 (s, 1H), 5.56 (s, 1H), 3.58 (dt, J=8.1, 4.4 Hz, 1H), 3.54-3.47 (m, 1H), 3.43 (dd, J=2.8, 1.4 Hz, 1H), 3.25 (d, J=5.5 Hz, 1H), 3.19 (d, J=17.8 Hz, 1H), 3.10 (dd, J=11.7, 5.2 Hz, 1H), 2.77 (dt, J=17.8, 2.6 Hz, 1H), 2.71 (dd, J=16.9, 4.1 Hz, 1H), 2.61 (ddt, J=12.6, 5.5, 2.8 Hz, 1H), 2.53 (dd, J=16.9, 2.5 Hz, 1H), 2.20 (d, J=12.6 Hz, 1H), 1.96-1.76 (m, 4H);

¹³C NMR (125 MHz, CDCl₃) δ 206.7, 201.3, 167.2, 146.8, 121.1, 59.0, 50.1, 49.3, 47.7, 47.6, 36.6, 36.6, 26.8, 23.4;

HRMS (ESI): m/z Calc. for C₁₄H₁₈NO₃ [M+H]⁺: 248.1281. found: 248.1281.

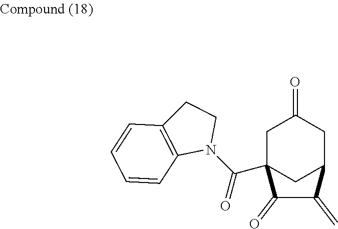 [SMILES: C=C1C(=O)[C@@]2(C(=O)N3CCc4ccccc43)CC(=O)C[C@@H]1C2]

Compound 18, white solid, yield: 47%.

¹H NMR (500 MHz, CDCl₃) δ 8.22 (d, J=8.1 Hz, 1H), 7.24-7.16 (m, 2H), 7.14-6.98 (m, 1H), 6.22 (s, 1H), 5.63 (s, 1H), 3.86 (td, J=9.6, 5.0 Hz, 1H), 3.80-3.71 (m, 1H), 3.50 (s, 1H), 3.28 (d, J=17.6 Hz, 1H), 3.17-3.05 (m, 2H), 2.95-2.82 (m, 1H), 2.83-2.71 (m, 2H), 2.58 (dd, J=16.8, 2.4 Hz, 1H), 2.30 (d, J=12.7 Hz, 1H);

¹³C NMR (125 MHz, CDCl₃) δ 206.3, 200.8, 167.1, 146.6, 143.6, 131.4, 127.7, 124.7, 124.6, 121.7, 118.3, 60.1, 50.2, 49.6, 49.4, 37.1, 36.7, 29.1;

HRMS (ESI): m/z Calc. for C₁₈H₁₈NO₃ [M+H]⁺: 296.1281. found: 296.1280.

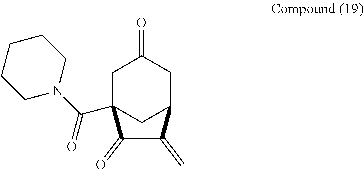 [SMILES: C=C1C(=O)[C@@]2(C(=O)N3CCCCC3)CC(=O)C[C@@H]1C2]

Compound 19, white solid, yield: 44%.

¹H NMR (500 MHz, CDCl₃) δ 6.17 (s, 1H), 5.58 (s, 1H), 3.88-3.32 (m, 3H), 3.34-2.93 (m, 3H), 2.87-2.63 (m, 2H), 2.59-2.41 (m, 2H), 2.21 (d, J=12.5 Hz, 1H), 1.76-1.31 (m, 6H);

¹³C NMR (125 MHz, CDCl₃) δ 206.8, 200.7, 167.4, 146.5, 121.4, 58.1, 50.8, 49.5, 44.2, 37.5, 36.6, 25.6, 24.5;

HRMS (ESI): m/z Calc. for C₁₅H₂₀NO₃ [M+H]⁺: 262.1438. found: 262.1439.

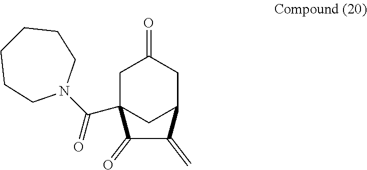 [SMILES: C=C1C(=O)[C@@]2(C(=O)N3CCCCCC3)CC(=O)C[C@@H]1C2]

Compound 20, white solid, yield: 31%.

¹H NMR (500 MHz, CDCl₃) δ 6.15 (s, 1H), 5.57 (s, 1H), 3.45 (d, J=28.7 Hz, 3H), 3.18 (d, J=17.8 Hz, 1H), 3.13-3.04 (m, 2H), 2.81-2.72 (m, 1H), 2.69 (dd, J=16.9, 4.0 Hz, 1H), 2.64-2.56 (m, 1H), 2.51 (dd. J=16.8, 2.4 Hz, 1H), 2.21 (d, J=12.7 Hz, 1H), 1.75 (s, 1H), 1.70-1.58 (m, 5H), 1.56-1.43 (m, 2H);

¹³C NMR (125 MHz, CDCl₃) δ 206.9, 200.9, 168.4, 146.7, 121.2, 58.4, 51.0, 49.6, 48.6, 47.5, 37.4, 36.7, 29.0, 28.7, 26.9, 25.3;

HRMS (ESI): m/z Calc. for C₁₆H₂₂NO₃ [M+H]⁺: 276.1594. found: 276.1595.

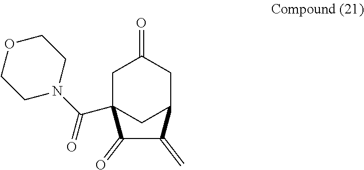 [SMILES: C=C1C(=O)[C@@]2(C(=O)N3CCOCC3)CC(=O)C[C@@H]1C2]

Compound 21, white solid, yield: 38%.

¹H NMR (500 MHz, CDCl₃) δ 6.18 (d, J=1.2 Hz, 1H), 5.60 (d, J=1.1 Hz, 1H), 3.81-3.35 (m, 9H), 3.11 (d, J=17.7 Hz, 1H), 2.81-2.65 (m, 2H), 2.59-2.51 (m, 2H), 2.24 (d, J=12.4 Hz, 1H);

¹³C NMR (125 MHz, CDCl₃) δ 206.2, 200.9, 167.8, 146.2, 122.0, 66.6, 58.2, 50.3, 49.3, 37.4, 36.4;

HRMS (ESI): m/z Calc. for C₁₄H₁₈NO₄ [M+H]⁺: 264.1230. found: 264.1231.

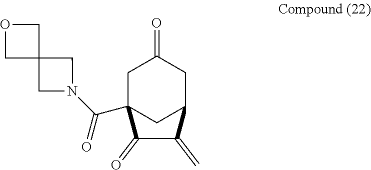 [SMILES: C=C1C(=O)[C@@]2(C(=O)N3CC4(COC4)C3)CC(=O)C[C@@H]1C2]

Compound 22, white solid, yield: 31%.

¹H NMR (500 MHz, CDCl₃) δ 6.16 (s, 1H), 5.57 (s, 1H), 4.78 (s, 4H), 4.42 (s, 1H), 4.21 (s, 3H), 3.48-3.39 (m, 1H), 2.95 (d, J=17.6 Hz, 1H), 2.79-2.64 (m, 2H), 2.55 (tdt, J=9.1, 4.9, 2.5 Hz, 2H), 2.16 (d, J=12.7 Hz, 1H);

¹³C NMR (125 MHz, CDCl₃) δ 205.9, 201.0, 168.5, 146.5, 121.5, 81.0, 80.7, 61.4, 58.7, 58.1, 48.9, 48.5, 38.6, 36.4, 36.1;

HRMS (ESI): m/z Calc. for C₁₅H₁₈NO₄ [M+H]⁺: 276.1230. found: 276.1232.

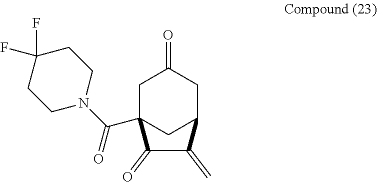 [SMILES: C=C1C(=O)[C@@]2(C(=O)N3CCC(F)(F)CC3)CC(=O)C[C@@H]1C2]

Compound 23, white solid, yield: 33%.

¹H NMR (500 MHz, CDCl₃) δ 6.19 (s, 1H), 5.62 (s, 1H), 3.60-3.24 (m, 5H), 3.11 (d, J=17.7 Hz, 1H), 2.89-2.68 (m, 2H), 2.63-2.44 (m, 2H), 2.25 (d, J=12.5 Hz, 1H), 2.09-1.88 (m, 4H);

¹³C NMR (125 MHz, CDCl₃) δ 206.0, 200.7, 167.8, 146.1, 122.17, 121.7 (t, J=241.3 Hz), 58.3, 50.5, 49.3, 37.6, 36.4, 33.8 (t, J=23.8 Hz);

¹⁹F NMR (470 MHz, CDCl₃) δ −96.89 (d, J=210.6 Hz), −100.82 (d, J=197.3 Hz);

HRMS (ESI): m/z Calc. for C₁₅H₁₇F₂NO₃ [M+H]⁺: 298.1249. found: 298.1251.

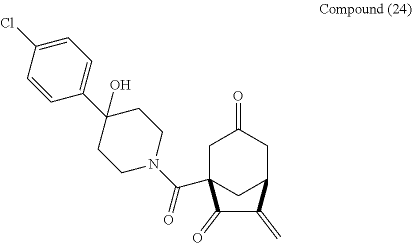 [SMILES: C=C1C(=O)[C@@]2(C(=O)N3CCC(O)(c4ccc(Cl)cc4)CC3)CC(=O)C[C@@H]1C2]

Compound 24, white solid, yield: 43%.

¹H NMR (500 MHz, CDCl₃) δ 7.39 (d, J=8.6 Hz, 2H), 7.30 (d, J=8.6 Hz, 2H), 6.16 (s, 1H), 5.59 (s, 1H), 4.57 (s, 1H), 3.51-3.03 (m, 5H), 2.80-2.66 (m, 2H), 2.61-2.44 (m, 2H), 2.21 (d, J=12.1 Hz, 2H), 2.09-1.71 (m, 4H);

¹³C NMR (125 MHz, CDCl₃) δ 206.7, 200.9, 167.5, 146.3, 146.1, 133.3, 128.7, 126.2, 121.8, 71.6, 58.0, 50.7, 49.4, 43.1, 39.6, 38.9, 38.1, 37.4, 36.6;

HRMS (ESI): m/z Calc. for C₂₁H₂₃ClNO₄ [M+H]⁺: 388.1310. found: 388.1312.

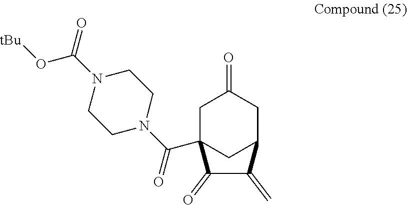 [SMILES: C=C1C(=O)[C@@]2(C(=O)N3CCN(C(=O)OC(C)(C)C)CC3)CC(=O)C[C@@H]1C2]

Compound 25, white solid, yield: 41%.

¹H NMR (500 MHz, CDCl₃) δ 6.18 (s, 1H), 5.60 (s, 1H), 3.66-3.00 (m, 10H), 2.86-2.64 (m, 2H), 2.61-2.39 (m, 2H), 2.23 (d, J=12.4 Hz, 1H), 1.53-1.34 (m, 9H);

¹³C NMR (125 MHz, CDCl₃) δ 206.1, 200.9, 167.8, 154.6, 146.2, 122.0, 80.4, 58.3, 50.4, 49.3, 43.8, 42.8, 37.4, 36.4, 28.4;

HRMS (ESI): m/z Calc. for C₁₉H₂₆N₂NaO₅ [M+Na]⁺: 385.1734. found: 385.1734.

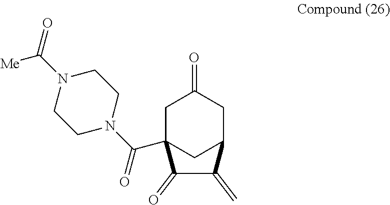 [SMILES: C=C1C(=O)[C@@]2(C(=O)N3CCN(C(C)=O)CC3)CC(=O)C[C@@H]1C2]

Compound 26, colorless oil, yield: 42%.

¹H NMR (500 MHz, CDCl₃) δ 6.19 (s, 1H), 5.62 (s, 1H), 3.72-3.35 (m, 9H), 3.10 (d, J=17.7 Hz, 1H), 2.85-2.66 (m, 2H), 2.64-2.50 (m, 2H), 2.24 (d, J=12.6 Hz, 1H), 2.09 (s, 3H);

¹³C NMR (125 MHz, CDCl₃) δ 206.0, 200.9, 169.3, 167.9, 146.1, 122.2, 58.3, 50.3, 49.3, 46.0, 41.0, 37.5, 36.3, 21.5;

HRMS (ESI): m/z Calc. for C₁₆H₂₁N₂O₄ [M+H]⁺: 305.1496. found: 305.1497.

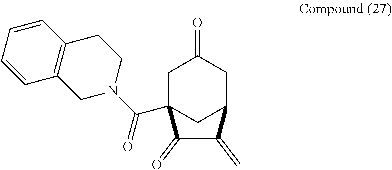 [SMILES: C=C1C(=O)[C@@]2(C(=O)N3CCc4ccccc4C3)CC(=O)C[C@@H]1C2]

Compound 27, colorless oil, yield: 14%.

¹H NMR (500 MHz, CDCl₃) δ 7.18 (d, J=4.1 Hz, 2H), 7.15-7.11 (m, 2H), 6.24 (d, J=1.2 Hz, 1H), 5.64 (d, J=1.0 Hz, 1H), 4.89-3.99 (m, 3H), 3.51-3.46 (m, 2H), 3.20 (d, J=17.7 Hz, 1H), 2.96-2.87 (m, 1H), 2.87-2.74 (m, 2H), 2.73 (dd, J=16.9, 4.1 Hz, 1H), 2.61-2.52 (m, 2H), 2.26 (d, J=12.7 Hz, 1H);

¹³C NMR (125 MHz, CDCl₃) δ 206.5, 200.8, 168.0, 146.6, 132.6, 128.8, 126.7, 121.7, 58.4, 50.6, 49.4, 37.5, 36.6, 28.7;

HRMS (ESI): m/z Calc. for C₁₉H₂₀NO₃ [M+H]⁺: 310.1437. found: 310.1438.

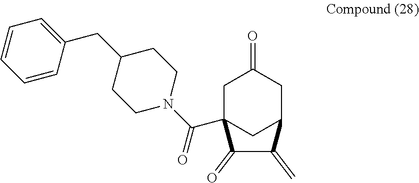 [SMILES: C=C1C(=O)[C@@]2(C(=O)N3CCC(Cc4ccccc4)CC3)CC(=O)C[C@@H]1C2]

Compound 28, colorless oil, yield: 46%.

¹H NMR (500 MHz, CDCl₃) δ 7.30-7.23 (m, 2H), 7.19 (t, J=7.3 Hz, 1H), 7.12 (d, J=7.2 Hz, 2H), 6.16 (s, 1H), 5.57 (s, 1H), 3.48-3.29 (m, 1H), 3.21-3.07 (m, 2H), 2.84-2.40 (m, 9H), 2.22 (s, 1H), 1.90-1.58 (m, 3H), 1.18 (td, J=12.5, 3.8 Hz, 2H);

¹³C NMR (125 MHz, CDCl₃) δ 206.7, 200.7, 167.4, 146.5, 140.0, 129.2, 128.4, 126.2, 121.5, 58.2, 50.8, 49.5, 42.9, 38.4, 37.5, 36.6, 32.1, 29.8;

HRMS (ESI): m/z Calc. for C₂₂H₂₆NO₃ [M+H]⁺: 352.1907. found: 352.1906.

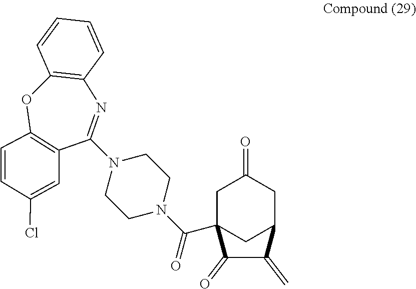 [SMILES: C=C1C(=O)[C@@]2(C(=O)N3CCN(C4=Nc5ccccc5Oc5ccc(Cl)cc54)CC3)CC(=O)C[C@@H]1C2]

Compound 29, colorless oil, yield: 26%.

¹H NMR (500 MHz, CDCl₃) δ 7.40 (dd, J=8.7, 2.6 Hz, 1H), 7.30 (d, J=2.6 Hz, 1H), 7.18 (d, J=8.7 Hz, 1H), 7.15-7.06 (m, 3H), 7.01 (td, J=7.5, 1.9 Hz, 1H), 6.21 (d, J=1.1 Hz, 1H), 5.62 (s, 1H), 3.66-3.36 (m, 9H), 3.15 (d, J=17.7 Hz, 1H), 2.80 (d, J=17.7 Hz, 1H), 272 (dd, J=16.9, 4.0 Hz, 1H), 2.55 (d, J=16.4 Hz, 2H), 2.26 (d, J=12.8 Hz, 1H);

¹³C NMR (125 MHz, CDCl₃) δ 206.2, 200.9, 167.9, 159.5, 158.7, 151.9, 146.2, 139.8, 1323.0, 130.6, 129.0, 127.2, 126.0, 125.2, 124.8, 123.0, 122.1, 120.3, 58.3, 50.4, 49.4, 47.3, 37.5, 36.5;

HRMS (ESI): m/z Calc. for C₂₇H₂₅ClN₃O₄ [M+H]⁺: 490.1528. found: 490.1527.

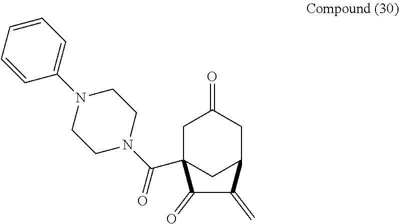 [SMILES: C=C1C(=O)[C@@]2(C(=O)N3CCN(c4ccccc4)CC3)CC(=O)C[C@@H]1C2]

Compound 30, colorless oil, yield: 15%.

¹H NMR (500 MHz, CDCl₃) δ 7.32-7.27 (m, 2H), 6.91 (t, J=7.6 Hz, 3H), 6.22 (s, 1H), 5.63 (s, 1H), 3.93-3.38 (m, 5H), 3.22-3.09 (m, 5H), 2.86-2.79 (m, 1H), 2.73 (dd, J=16.7, 4.0 Hz, 1H), 2.64-2.55 (m, 2H), 2.27 (d, J=12.6 Hz, 1H);

¹³C NMR (125 MHz, CDCl₃) δ 206.3, 200.9, 167.7, 146.4, 129.4, 122.0, 120.8, 116.9, 58.3, 50.5, 49.4, 37.6, 36.5;

HRMS (ESI): m/z Calc. for C₂₀H₂₂N₂O₃ [M+H]⁺: 339.1703. found: 339.1705.

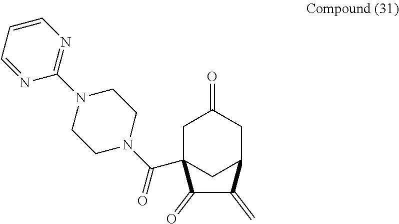 [SMILES: C=C1C(=O)[C@@]2(C(=O)N3CCN(c4ncccn4)CC3)CC(=O)C[C@@H]1C2]

Compound 31, colorless oil, yield: 40%.

¹H NMR (500 MHz, CDCl₃) δ 8.31 (d, J=4.7 Hz, 2H), 6.53 (s, 1H), 6.20 (s, 1H), 5.62 (s, 1H), 3.88-3.62 (m, 6H), 3.47-3.28 (m, 3H), 3.15 (d, J=17.7 Hz, 1H), 2.80 (d, J=17.7 Hz, 1H), 2.72 (dd, J=16.9, 3.9 Hz, 1H), 2.66-2.52 (m, 2H), 2.26 (d, J=12.7 Hz, 1H);

¹³C NMR (125 MHz, CDCl₃) δ 206.3, 200.9, 167.9, 161.7, 157.9, 146.4, 121.9, 110.7, 58.3, 50.5, 49.4, 43.5, 37.6, 36.5;

HRMS (ESI): m/z Calc. for C₁₈H₂₁N₄O₃ [M+H]⁺: 341.1608. found: 341.1608.

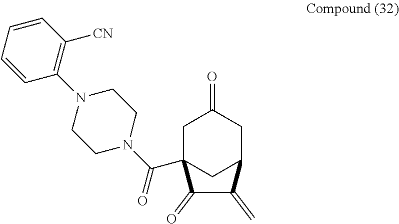 [SMILES: C=C1C(=O)[C@@]2(C(=O)N3CCN(c4ccccc4C#N)CC3)CC(=O)C[C@@H]1C2]

Compound 32, white solid, yield: 21%.

¹H NMR (500 MHz, CDCl₃) δ 7.58 (dd, J=7.7, 1.4 Hz, 1H), 7.54-7.42 (m, 1H), 7.06 (t, J=7.4 Hz, 1H), 7.01 (d, J=8.3 Hz, 1H), 6.19 (s, 1H), 5.63 (s, 1H), 4.15 (s, 1H), 3.67-3.39 (m, 4H), 3.25-2.99 (m, 5H), 2.80 (d, J=17.8 Hz. 1H), 2.73 (dd, J=16.8, 4.0 Hz, 1H), 2.70-2.49 (m, 2H), 2.26 (d, J=12.7 Hz, 1H);

¹³C NMR (125 MHz, CDCl₃) δ 206.3, 201.1, 167.8, 155.2, 146.2, 134.4, 134.1, 122.8, 122.1, 119.2, 118.3, 106.7, 58.3, 51.3, 50.5, 49.4, 37.4, 36.5;

HRMS (ESI): m/z Calc. for C₂₁H₂₂N₃O₃ [M+H]⁺: 364.1656. found: 364.1658.

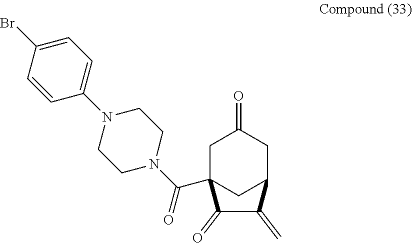 [SMILES: C=C1C(=O)[C@@]2(C(=O)N3CCN(c4ccc(Br)cc4)CC3)CC(=O)C[C@@H]1C2]

Compound 33, white solid, yield: 22%.

¹H NMR (500 MHz, CDCl₃) δ 7.34 (d, J=8.9 Hz, 2H), 6.76 (d, J=8.9 Hz, 2H), 6.20 (s, 1H), 5.62 (s, 1H), 3.72-3.38 (m, 5H), 3.13 (d, J=17.7 Hz, 5H), 2.79 (d, J=17.7 Hz, 1H), 2.72 (dd, J=16.8, 3.9 Hz, 1H), 2.63-2.50 (m, 2H), 2.26 (d, J=12.7 Hz, 1H);

¹³C NMR (125 MHz, CDCl₃) δ 206.2, 200.9, 167.7, 149.9, 146.3, 132.1, 122.0, 118.4, 112.9, 58.3, 50.4, 49.3, 49.1, 37.5, 36.4;

HRMS (ESI): m/z Calc. for C₂₀H₂₂BrN₂O₃ [M+H]⁺: 417.0808. found: 417.0807.

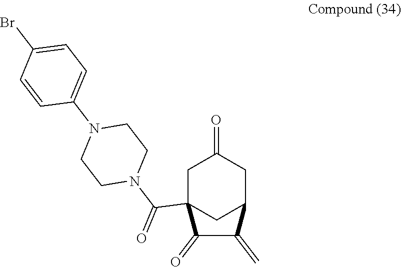 [SMILES: C=C1C(=O)[C@@]2(C(=O)N3CCN(c4ccc(Br)cc4)CC3)CC(=O)C[C@@H]1C2]

Compound 34, white solid, yield: 72%.

¹H NMR (500 MHz, CDCl₃) δ 7.32 (d, J=9.0 Hz, 2H), 6.75 (d, J=9.0 Hz, 2H), 5.00 (s, 1H), 4.88 (s, 1H), 3.68 (d, J=3.5 Hz, 4H), 3.09 (t, J=5.1 Hz, 4H), 2.96 (d, J=4.5 Hz, 1H), 2.76-2.57 (m, 4H), 2.52 (dd, J=16.1, 4.0 Hz, 1H), 2.43-2.33 (m, 2H), 2.17 (dd, J=12.5, 1.9 Hz, 1H);

¹³C NMR (125 MHz, CDCl₃) δ 208.5, 172.7, 151.2, 149.9, 132.2, 118.3, 113.0, 108.9, 51.6, 51.3, 50.0, 49.6, 42.2, 40.5, 40.4;

HRMS (ESI): m/z Calc. for C₂₀H₂₄BrN₂O₂ [M+H]⁺: 403.1016. found: 403.1016.

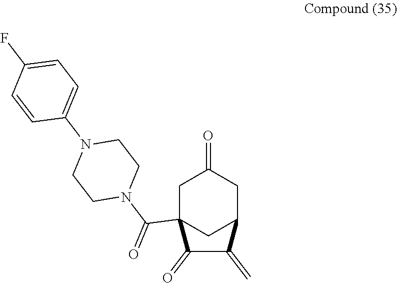 [SMILES: C=C1C(=O)[C@@]2(C(=O)N3CCN(c4ccc(F)cc4)CC3)CC(=O)C[C@@H]1C2]

Compound 35, colorless oil, yield: 29%.

¹H NMR (500 MHz, CDCl₃) δ 6.97 (t, J=8.6 Hz, 2H), 6.90-6.79 (m, 2H), 6.21 (s, 1H), 5.63 (s, 1H), 3.71-3.34 (m, 5H), 3.15 (d, J=17.7 Hz, 1H), 3.12-3.02 (m, 4H), 2.80 (d, J=17.7 Hz, 1H), 2.73 (dd, J=16.7, 3.9 Hz, 1H), 2.64-2.52 (m, 2H), 2.27 (d, J=12.6 Hz, 1H);

¹³C NMR (125 MHz, CDCl₃) δ 206.3, 200.9, 167.7, 157.8 (d, J=238.8 Hz), 147.6, 146.3, 122.0, 118.8 (d, J=3.8 Hz), 115.8 (d, J=11.3 Hz), 58.3, 50.5, 50.4, 49.4, 37.6, 36.4;

¹⁹F NMR (470 MHz, CDCl₃) δ −124.27;

HRMS (ESI): m/z Calc. for C₂₀H₂₂FN₂O₃ [M+H]⁺: 357.1609. found: 357.1611.

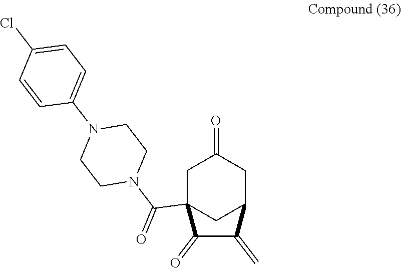 [SMILES: C=C1C(=O)[C@@]2(C(=O)N3CCN(c4ccc(Cl)cc4)CC3)CC(=O)C[C@@H]1C2]

Compound 36, white solid, yield: 31%.

¹H NMR (500 MHz, CDCl₃) δ 7.21 (d, J=9.0 Hz, 2H), 6.82 (d, J=9.0 Hz, 2H), 6.21 (s, 1H), 5.63 (s, 1H), 3.82-3.39 (m, 5H), 3.25-2.97 (m, 5H), 2.80 (d, J=17.7 Hz, 1H), 2.73 (dd, J=16.8, 4.0 Hz, 1H), 2.66-2.48 (m, 2H), 2.26 (d, J=12.7 Hz, 1H);

¹³C NMR (125 MHz, CDCl₃) δ 206.2, 200.9, 167.7, 149.5, 146.3, 129.2, 125.7, 122.0, 118.0, 58.3, 50.4, 49.4, 37.5, 36.4;

HRMS (ESI): m/z Calc. for C₂₀H₂₂ClN₂O₃ [M+H]⁺: 373.1313. found: 373.1315.

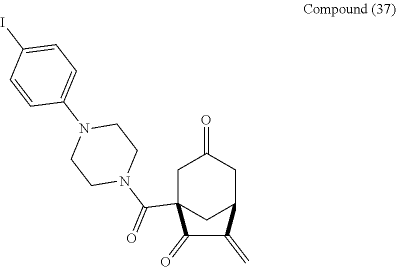 [SMILES: C=C1C(=O)[C@@]2(C(=O)N3CCN(c4ccc(I)cc4)CC3)CC(=O)C[C@@H]1C2]

Compound 37, white solid, yield: 22%.

¹H NMR (500 MHz, CDCl₃) δ 7.53 (d, J=8.8 Hz, 2H), 6.67 (d, J=8.5 Hz, 2H), 6.21 (s, 1H), 5.63 (s, 1H), 3.90-3.56 (m, 3H), 3.52-3.40 (m, 2H), 3.21-3.09 (m, 5H), 2.80 (d, J=17.7 Hz, 1H), 2.73 (dd, J=17.0, 4.1 Hz, 1H), 2.66-2.50 (m, 2H), 2.26 (d, J=12.9 Hz, 1H);

¹³C NMR (125 MHz, CDCl₃) δ 206.2, 200.9, 167.7, 150.5, 146.3, 138.1, 122.0, 118.8, 82.8, 58.3, 50.4, 49.4, 49.0, 37.6, 36.4;

HRMS (ESI): m/z Calc. for C₂₀H₂₂IN₂O₃ [M+H]⁺: 465.0670. found: 465.0671.

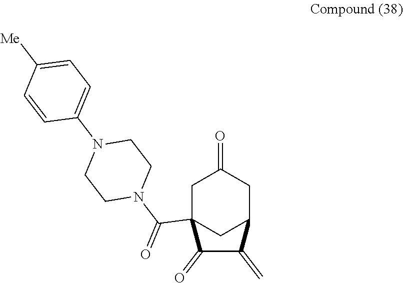 [SMILES: C=C1C(=O)[C@@]2(C(=O)N3CCN(c4ccc(C)cc4)CC3)CC(=O)C[C@@H]1C2]

Compound 38, colorless oil, yield: 31%.

¹H NMR (500 MHz, CDCl₃) δ 7.08 (d, J=8.3 Hz, 2H), 6.84 (d, J=8.0 Hz, 2H), 6.21 (s, 1H), 5.62 (s, 1H), 3.89-3.43 (m, 5H), 3.22-3.02 (m, 5H), 2.87-2.78 (m, 1H), 2.73 (dd, J=16.7, 4.0 Hz, 1H), 2.65-2.51 (m, 2H), 2.43-2.21 (m, 4H);

¹³C NMR (125 MHz, CDCl₃) δ 206.3, 200.9, 167.7, 148.7, 146.4, 129.9, 125.7, 121.9, 117.2, 58.3, 50.5, 50.0, 49.4, 37.6, 36.5, 20.6;

HRMS (ESI): m/z Calc. for C₂₁H₂₅IN₂O₃ [M+H]⁺: 353.1860. found: 353.1862.

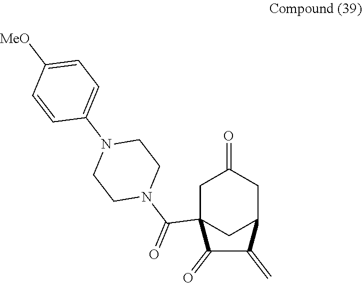 [SMILES: C=C1C(=O)[C@@]2(C(=O)N3CCN(c4ccc(OC)cc4)CC3)CC(=O)C[C@@H]1C2]

Compound 39, colorless oil, yield: 13%.

¹H NMR (500 MHz, CDCl₃) δ 6.89 (d, J=8.3 Hz, 2H), 6.84 (d, J=9.0 Hz, 2H), 6.21 (s, 1H), 5.62 (s, 1H), 4.03-3.56 (m, 6H), 3.55-3.32 (m, 2H), 3.15 (d, J=17.7 Hz, 1H), 3.29-3.08 (m, 4H), 2.90-2.78 (m, 1H), 2.73 (dd, J=16.7, 4.0 Hz, 1H), 2.71-2.47 (m, 2H), 2.27 (d, J=12.6 Hz, 1H);

¹³C NMR (125 MHz, CDCl₃) δ 206.3, 200.9, 167.7, 154.6, 146.4, 145.1, 121.9, 119.1, 114.7, 58.3, 55.7, 50.9, 50.5, 49.4, 37.6, 36.5;

HRMS (ESI): m/z Calc. for C₂₁H₂₅N₂O₄ [M+H]⁺: 369.1808. found: 369.1810.

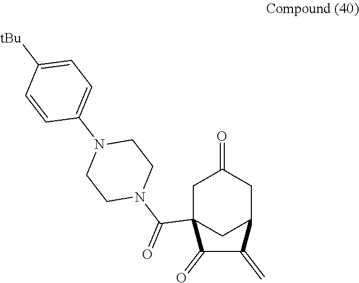 [SMILES: C=C1C(=O)[C@@]2(C(=O)N3CCN(c4ccc(C(C)(C)C)cc4)CC3)CC(=O)C[C@@H]1C2]

Compound 40, colorless oil, yield: 12%.

¹H NMR (500 MHz, CDCl₃) δ 7.30 (d, J=8.6 Hz. 2H), 6.88 (s, 2H), 6.22 (s, 1H), 5.63 (s, 1H), 3.79-3.41 (m, 5H), 3.21-3.09 (m, 5H), 2.81 (d, J=17.8 Hz, 1H), 2.73 (dd, J=16.7, 4.0 Hz, 1H), 2.64-2.49 (m, 2H), 2.27 (d, J=12.6 Hz, 1H), 1.29 (s, 9H);

¹³C NMR (125 MHz, CDCl₃) δ 206.3, 200.9, 167.7, 148.5, 146.4, 126.2, 121.9, 116.6, 58.3, 50.5, 49.7, 49.4, 37.6, 36.5, 34.2, 31.5;

HRMS (ESI): m/z Calc. for C₂₄H₃₁IN₂O₃ [M+H]⁺:395.2329. found: 395.2331.

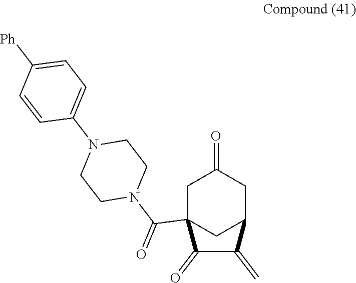 [SMILES: C=C1C(=O)[C@@]2(C(=O)N3CCN(c4ccc(-c5ccccc5)cc4)CC3)CC(=O)C[C@@H]1C2]

Compound 41, white solid, yield: 11%.

¹H NMR (500 MHz, CDCl₃) δ 7.54 (t, J=8.0 Hz, 4H), 7.41 (t, J=7.6 Hz, 2H), 7.30 (t, J=7.3 Hz, 1H), 7.02 (s, 2H), 6.23 (s, 1H), 5.64 (s, 1H), 3.92-3.55 (m, 4H), 3.48 (s, 1H), 3.32-3.22 (m, 4H), 3.16 (d, J=17.7 Hz, 1H), 2.82 (d, J=17.8 Hz, 1H), 2.74 (dd, J=16.7, 3.9 Hz, 1H), 2.66-2.51 (m, 2H), 2.28 (d, J=12.6 Hz, 1H);

¹³C NMR (125 MHz, CDCl₃) δ 206.2, 201.0, 167.7, 146.3, 140.8, 128.9, 128.1, 126.8, 122.1, 117.0, 58.4, 50.5, 49.4, 37.6, 36.5;

HRMS (ESI): m/z Calc. for C₂₆H₂₇IN₂O₃ [M+H]⁺:415.2016. found: 415.2019.

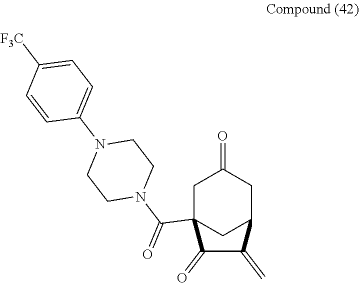 [SMILES: C=C1C(=O)[C@@]2(C(=O)N3CCN(c4ccc(C(F)(F)F)cc4)CC3)CC(=O)C[C@@H]1C2]

Compound 42, colorless oil, yield: 36%.

¹H NMR (500 MHz, CDCl₃) δ 7.49 (d, J=8.7 Hz, 2H), 6.91 (d, J=8.6 Hz, 2H), 6.22 (s, 1H), 5.64 (s, 1H), 3.82-3.41 (m, 5H), 3.32-3.23 (m, 4H), 3.14 (d, J=17.7 Hz, 1H), 2.81 (d, J=17.7 Hz, 1H), 2.74 (dd, J=16.9, 3.9 Hz, 1H), 2.67-2.52 (m, 2H), 2.28 (d, J=12.7 Hz, 1H);

¹³C NMR (125 MHz, CDCl₃) δ 206.1, 200.9, 167.8, 153.0, 146.3, 126.7 (d, J=1.3 Hz), 124.7 (q, J=134.4 Hz), 122.1, 121.6 (d, J=16.3 Hz), 115.3, 58.4, 50.4, 49.4, 48.1, 37.6, 36.4;

¹⁹F NMR (470 MHz, CDCl₃) δ −62.67;

HRMS (ESI): m/z Calc. for C₂₁H₂₂F₃N₂O₃ [M+H]⁺: 407.1577. found: 407.1580.

### Biological Evaluation:

### Kinetic Characterization of SHP2 Inactivation

Inhibitors were evaluated for their ability to inhibit the reaction catalyzed by SHP2 using para-nitrophenyl phosphate (pNPP) as a substrate at pH 7 and 25° C. A 2× solution of inhibitor was diluted into DMG buffer (50 mM 3,3-dimethylglutarate pH 7.0, 1 mM EDTA, ionic strength adjusted to 150 mM using NaCl) was added to a clear flat-bottom 96-well plate (Corning Costar Assay Plate #9017), and serially diluted 1.5-fold, resulting in 100 μL volumes of varying inhibitor concentrations. To this was added a 4× solution of pNPP (12 mM) in DMG buffer. The reaction was initialized by the addition of a 4× solution of SHP2 (residues 224-528), resulting in a final solution of: inhibitor (100-0 μM), pNPP (3 mM), and SHP2 (100 nM). The plate was then monitored for a change in absorbance ab 405 nM using a CLARIOstar^{Plus} plate reader (BMG Labetch) over the course of an hour. The resulting progress curves for each inhibitor were imported into Graphpad Prism 9 (GraphPad Software, San Diego, CA (USA)), and a baseline correction analysis was completed for fitting purposes. Each curve was fit to the equation Y=V0×(1−e^{-kobs*t})/k_{obs}.^{[17.22]} The k_{obs} values were then plotted against inhibitor concentrations. If the resulting graph was linear, the k_{inact}/K_{I} value was determined by the slope of that line and the 95% confidence interval (95% CI). If the resulting graph was non-linear, the curve was fit to the equation Y=k_{inact}*X/(K_{I}+X).

### Time-Dependent IC₅₀ Determination

Inhibitors were evaluated for their ability to inhibit the reaction catalyzed by SHP2 using para-nitrophenyl phosphate (pNPP) as a substrate at pH 7 and 25° C. The reaction was started in a clear flat-bottom 96-well plate (Corning Costar Assay Plate #9017) by the addition of 25 μL of 4 μM PTP in DMG buffer to 25 μl of 2× inhibitor (final concentrations=2 μM PTP, 100-0 μM inhibitor). Reactions were incubated for 1 or 10 minutes before transferring 4 μL into 196 μL of 10.1 mM pNPP in DMG buffer (final concentration=40 nM PTP, 10 mM pNPP). These wells were then allowed to react for 10, 45, or 3 minutes for SHP1 and SHP2, LYP, PTP1B respectively before quenching with 50 μL of 5 M NaOH. Plates were then read for absorbance at 405 nm using a CLARIOstar^{Plus} plate reader (BMG Labetch). Data was imported into Graphpad Prism 9 (GraphPad Software, San Diego, CA (USA), and a baseline correction analysis was completed for fitting purposes by subtracting a well containing only 10 mM pNPP. IC₅₀ values were then determined using Graphpad Prism 9 using the DMSO control as 100% active.

### Electrophile Reactivity Assessment

To a 100 μL solution of 1 mM electrophile in Buffer A (150 mM Sodium Phosphate pH 7.5, 100 μM NaCl, 1 mM EDTA) was added 100 μL of 1 mM reduced glutathione in Buffer A in a clear flat-bottom 96-well plate (Corning Costar Assay Plate #9017). This reaction was held at 25° C. for 45 minutes before quenching with 50 μl of 9.25 mM DTNB in Buffer A. Plates were read at 412 nM using a CLARIOstar^{Plus} plate reader (BMG Labetch). Data was imported into Graphpad Prism 9 (GraphPad Software, San Diego, CA (USA)) for analysis, using the DMSO-treated control samples as 100% GSH remaining.

### Active Site Protection by Vanadate

A solution containing 500 μM Vanadate or DMSO, 4 μM SHP2, and 100 μM (10) or DMSO in DMG buffer (with no EDTA) was added to a clear flat-bottom 96-well plate (Corning Costar Assay Plate #9017). This was allowed to incubate for 10 minutes at 25° C. before transferring 3 μL to a new clear flat-bottom 96-well plate (Corning Costar Assay Plate #9017). To each well was added 297 μL of 10.1 mM pNPP in DMG buffer (with EDTA) and the plate Plates were then read for absorbance at 405 nm using a CLARIOstar^{Plus} plate reader (BMG Labetch). Data was imported into Graphpad Prism 9 (GraphPad Software, San Diego, CA (US)), and a baseline correction analysis was completed for fitting purposes.

### Molecular Modeling

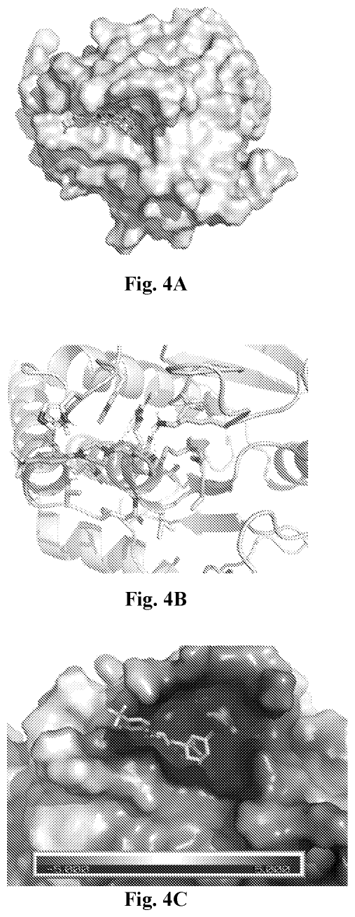

To structurally rationalize the experimentally observed SAR, a molecular modeling experiment was performed. Without any previously deposited covalently modified SHP2 crystal structures available, an unbiased docking approach was used to predict putative binding poses for compound (42) with SHP2. An unbiased docking of compound (42) in the active site of SHP2 (PDB ID 3O5X) was perform using CovDock (Schrödinger, LLC), with cysteine 459 selected as the nucleophilic residue in accordance with our data suggesting active site binding. The resulting docking was analyzed by cdock affinity, which enriches for the most likely poses. Four of the top 5 poses bound to SHP2 in a similar manner (FIG. 4A). The top scoring pose is predicted to form hydrogen bonds between the carbonyls of the bicyclic ring and sidechain amine of Lys 366, with an additional hydrogen bond predicted to form between the amide carbonyl of compound (42) and the sidechain guanidine of Arg 465. Van der Waals interactions are predicted to form between the bicyclic warhead of compound (42) and the SHP2 active site, with further Van der Waals interactions being predicted to form with the piperazine moiety. Further away from the active site, an additional pi-pi interaction is predicted to occur between the phenyl ring of compound (42) and His 426 (FIG. 4B). This pose is consistent with the experimentally observed SAR. Increasing ring size corresponded to increased compound efficacy, consistent with the Van der Waals interactions predicted from this pose; a larger ring size would fill this pocket more completely. Furthermore, addition of a phenyl ring in compound (30) to the piperazine core resulted in an increase in efficacy relative to the N-acetyl (compound (26)) or N-Boc analogs (compound (25)), corroborating with the predicted pi-pi interaction that is predicted from this model. Finally, the clear relationship between hydrophobicity on the para position of the phenyl ring and efficacy may be rationalized using this model. The solvent exposed nature of this group suggests that the additional hydrophobic nature of these substituents contributes to binding in a non-specific manner. Furthermore, the electrostatic potential of the SHP2 surface at this interface is positive (FIG. 4C), providing rationale behind the observed correlation between increased compound efficacy and increasing electronegativity at the para substitution on the phenyl ring, supporting the validity of this binding pose. However, it should be noted that the predicted interactions between the phenyl ring of compound (42) and SHP2 occur at the WPD loop; this motif is known to be dynamic in nature and plasticity is required for enzymatic catalysis.

The ligand-receptor pose resulting from docking was used to perform a sequence alignment with the 3 other PTPs used in this study: SHP1 (PTPN6), LYP (PTPN22), and PTP1B (PTPN1). Residues within 7 Å of the ligand were compared across the four PTPs to search for any nearby residues that may impart selectivity towards SHP2. Among the different residues near the ligand were His 426, Gly 427, Glu 508, and Ala 509 (FIG. 5). These residues are clustered around the phenyl ring of compound (42), suggesting that the various 4-substituted phenyl ring analogs may display varying degrees of selectivity towards SHP2. Compound (37) displayed an interesting selectivity profile towards SHP2, as previously discussed. The 4-iodo substituted compound (37) bioisostere of compound (42) is expected to bind in a similar manner according to the docking results (FIG. 5). Using this docking model, it is possible to structurally rationalize the experimentally observed selectivity profile. The relative level of selectivity towards SHP2 corresponds to the sequence homology at this peripheral pocket. Compound (37) is the least selective for SHP2 over SHP1 (4/4 identical residues), while it is more selective for SHP2 over both LYP and PTP1B (1/4 identical residues), further supporting the validity of the predicted binding pose.

Molecular modeling studies of compound (42) with SHP2 was performed using the previously published crystal structure of SHP2 (PDB ID 3O5X) within the Maestro suite, version 2021-3 (Schrödinger, LLC, New York, NY). The protein was prepared according to the automated protein preparation workflow at a pH of 7.0±1.0 to match the conditions used in the biochemical assays. Before minimization, cysteine 459 was manually ionized. Minimization was performed by converging heavy atoms to an RMSD of 0.3 Å using the OPLS4 force field, and all waters further than 5 Å away from heteroatoms and with fewer than 3 hydrogen bonds to non-waters were removed. Compound (42) was prepared using the LigPrep module. Ionization states were generated at pH 7.0±1.0 to match biochemical assay conditions, and all combinations of stereoisomers were created. Residue 459 was defined as the reactive residue, and the center of the grid was defined by the co-crystallized ligand of 3O5X. The reaction type was set to “Michael Addition”. “Pose prediction” docking mode was selected, and 10 output poses were generated per ligand. Poses were sorted by “cdock affinity”. The top scoring pose was exported to PyMol and manually inspected.

### Method for LC-MS.

To unfold protein and quench protein-ligand reactivity, 10 μL of treated or untreated 10 μM SHP2 was diluted in 40 μL of 80% (v/v) formic acid initially, followed by 150 μL of LC-MS buffer (50/50 water/acetonitrile with 0.1% formic acid). 20-25 pmol per sample was analyzed by LC/MS (Agilent 1260 Infinity II with a ZORBAX Rapid Resolution High Definition 300 Å Stable Bond C3, 2.1×100 mm, 1.8 μm column) attached to an Agilent 6129 quadrupole mass spectrometer in positive ion mode. The column was held at 45° C. Mobile solution A was 0.1% formic acid in water, and mobile phase B was 0.1% formic acid in acetonitrile. The gradient used was hold at 10% B for 5 min, increase linearly to 80% B for 15 min, and then hold at 80% B for 5 min. The mass data were collected at a range of 600-1500 m/z. Raw data were processed using MestReNova. For all samples, the deconvoluted mass was calculated by taking the m/z range of 730-1100 Da, and deconvoluted mass range from 36500-37200 Da. An abundance threshold of 10% and tolerance of 30 ppm was set for deconvolution. Unreacted SHP2's total deconvoluted mass was 36602±10 Da; for each deconvolution, the highest deconvoluted mass intensity was utilized as the mass pivot point to determine and identify reacted SHP2 adducts. To calculate the percent SHP2 reacted, the relative abundance intensity of reacted SHP2 was divided by the total relative abundance intensities of unreacted and reacted SHP2.

### Results

| | Unreacted | Reacted SHP2 | | | | |
|---|---|---|---|---|---|---|
| | SHP2 Total | Total | | | | Percent |
| | Deconvoluted | Deconvoluted | | | | Error |
| | Mass (Da) | Mass (Da) | | Expected | Found | Between |
| | (Relative | (Relative | | Ligand | Ligand | Expected |
| | Abundance | Abundance | % SHP2 | Adduct | Adduct | and |
| Ligand | Intensity in %) | Intensity in %) | Reacted | (Da) | (Da) | Observed |
| PVSN | 36602 | 36790 | 21% | 184 | 188 | 2.1% |
| | (100%) | (27%) | | | | |
| 10 | 36604 | 36810 | 34% | 208 | 206 | 0.9% |
| | (100%) | (53%) | | | | |
| 33 | 36602 | 37014 | 30% | 416 | 412 | 0.9% |
| | (100%) | (41%) | | | | |
| 42 | 36597 | 37007 | 43% | 406 | 410 | 1% |
| | (100%) | (78%) | | | | |
| 37 | 36604 | 37065 | 33% | 464 | 461 | 0.6% |
| | (100%) | (49%) | | | | |

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

It is intended that the scope of the present compounds, compositions, and methods be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A compound of formula (I) or a pharmaceutically acceptable salt, hydrate, tautomer, or optical isomer thereof: 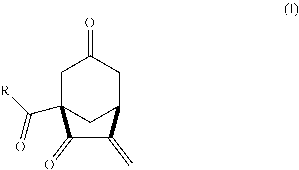
wherein
R is H, alkyl, alkenyl, alkynyl, cycloalkyl, acyl, heterocycloalkyl, aryl, heteroaryl, OR₁, or NR₁R₂, wherein R can be optionally substituted with at least one of —COOR₁, —SO₃R₁, halo, cyano, nitro, aryl, OR₁, and NR₁R₂;
wherein each R₁ and R₂ independently is H, alkyl, alkenyl, alkynyl, cycloalkyl, acyl, heterocycloalkyl, aryl, heteroaryl, OR₁, or NH₂; wherein each R₁ and R₂ can be optionally independently substituted with at least one of —COOR₁, —SO₃R₁, halo, cyano, nitro, cycloalkyl, aryl, heterocyclyl, OR₁, and NR₁R₂; or
R₁ and R₂, together with the nitrogen atom to which they are attached, form a 4- to 8-membered heterocyclic ring, which contains one or more heteroatoms selected from O, N, and S and is optionally substituted with at least one of halo, alkyl, cycloalkyl, heterocycloalkyl, aryl, heteroaryl, —COOR₁, —SO₃R₁, cyano, OR₁, COR₁, and CF₃, wherein alkyl, cycloalkyl, heterocycloalkyl, aryl, or heteroaryl can be optionally further substituted with halo, cyano, or substituted or unsubstituted aryl, wherein aryl can be substituted with at least one of halo, alkyl, cyano, phenyl, OCH₃, and CF₃.

2. A compound of formula (IA) or a pharmaceutically acceptable salt, hydrate, tautomer, or optical isomer thereof: 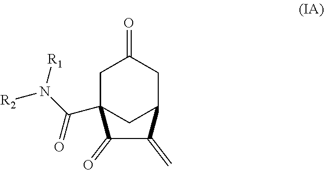
wherein each R₁ and R₂ independently is H, alkyl, alkenyl, alkynyl, cycloalkyl, acyl, heterocycloalkyl, aryl, heteroaryl, OR₁, or NH₂; wherein each R₁ and R₂ can be optionally independently substituted with at least one of —COOR₁, —SO₃R₁, halo, cyano, nitro, cycloalkyl, aryl, heterocyclyl, OR₁ and NR₁R₂;
or
R₁ and R₂, together with the nitrogen atom to which they are attached, form a 4- to 8-membered heterocyclic ring, which contains one or more heteroatoms selected from O, N, and S and is optionally substituted with at least one of halo, alkyl, cycloalkyl, heterocycloalkyl, aryl, heteroaryl, COOR₁, —SO₃R₁, cyano, OR₁, COR₁, and CF₃, wherein alkyl, cycloalkyl, heterocycloalkyl, aryl, and heteroaryl can be optionally further substituted with halo, cyano, or substituted or unsubstituted aryl, wherein aryl can be substituted with at least one of halo, alkyl, cyano, phenyl, OCH₃, and CF₃.

3. The compound of claim 2, wherein the compound of formula (IA) is: 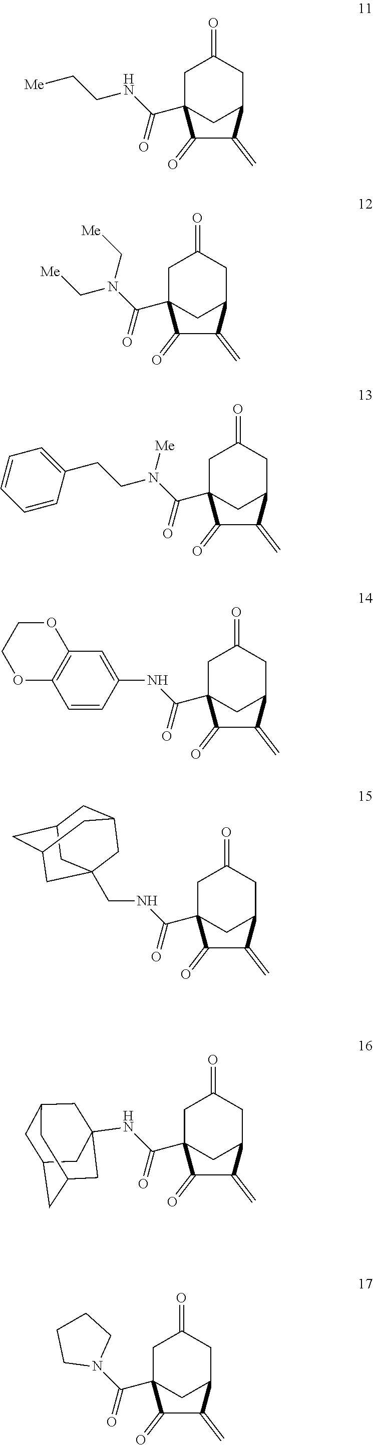 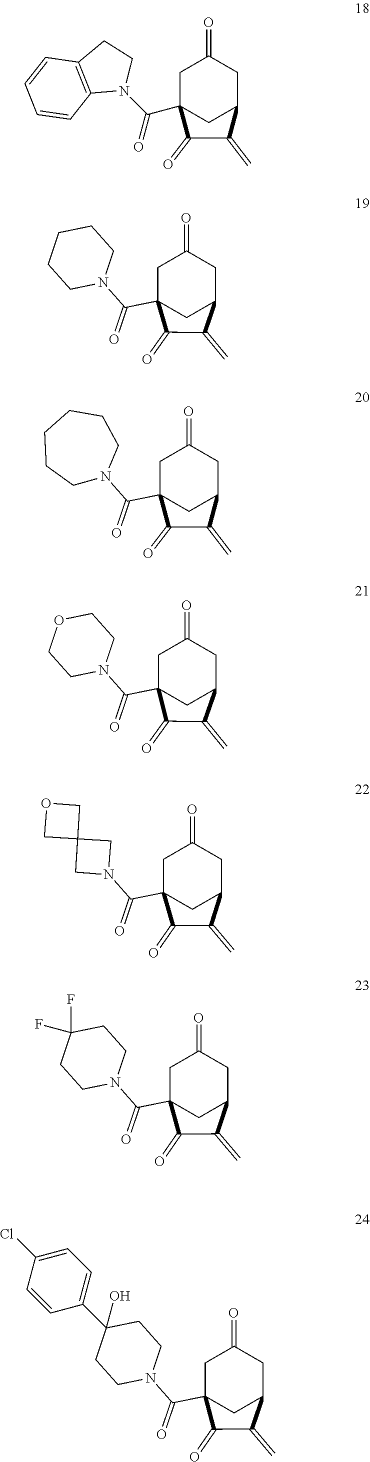 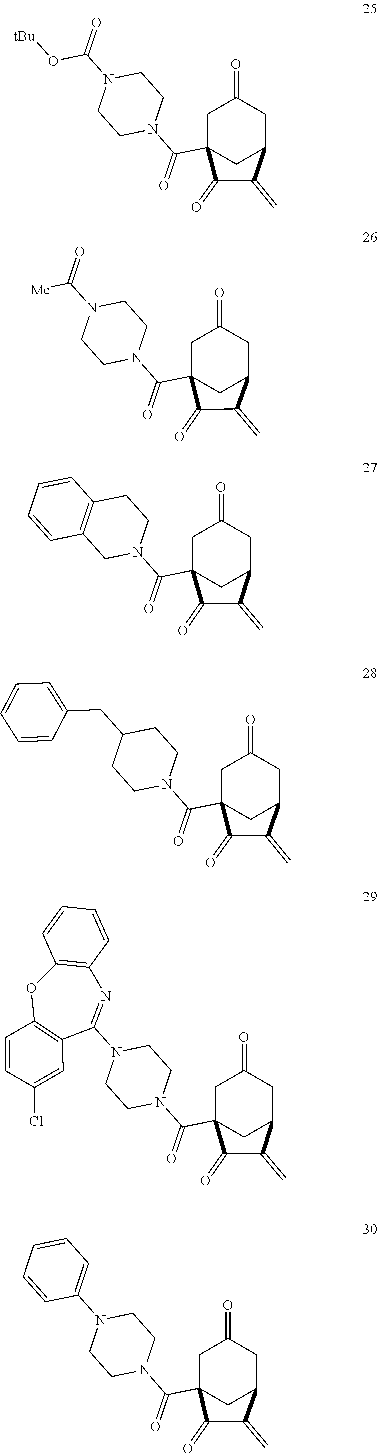 [SMILES: C=C1C(=O)[C@@]2(C(=O)N3CCN(C(=O)OC(C)(C)C)CC3)CC(=O)C[C@@H]1C2 ; C=C1C(=O)[C@@]2(C(=O)N3CCN(C(C)=O)CC3)CC(=O)C[C@@H]1C2 ; C=C1C(=O)[C@@]2(C(=O)N3CCc4ccccc4C3)CC(=O)C[C@@H]1C2 ; C=C1C(=O)[C@@]2(C(=O)N3CCC(Cc4ccccc4)CC3)CC(=O)C[C@@H]1C2 ; C=C1C(=O)[C@@]2(C(=O)N3CCN(C4=Nc5ccccc5Oc5ccc(Cl)cc54)CC3)CC(=O)C[C@@H]1C2 ; C=C1C(=O)[C@@]2(C(=O)N3CCN(c4ccccc4)CC3)CC(=O)C[C@@H]1C2] 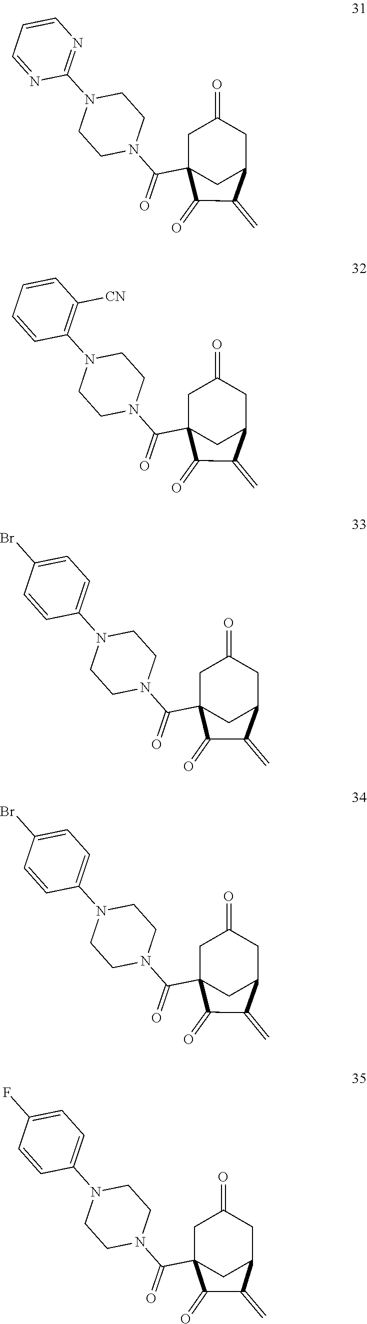 [SMILES: C=C1C(=O)[C@@]2(C(=O)N3CCN(c4ncccn4)CC3)CC(=O)C[C@@H]1C2 ; C=C1C(=O)[C@@]2(C(=O)N3CCN(c4ccccc4C#N)CC3)CC(=O)C[C@@H]1C2 ; C=C1C(=O)[C@@]2(C(=O)N3CCN(c4ccc(Br)cc4)CC3)CC(=O)C[C@@H]1C2 ; C=C1C(=O)[C@@]2(C(=O)N3CCN(c4ccc(Br)cc4)CC3)CC(=O)C[C@@H]1C2 ; C=C1C(=O)[C@@]2(C(=O)N3CCN(c4ccc(F)cc4)CC3)CC(=O)C[C@@H]1C2] 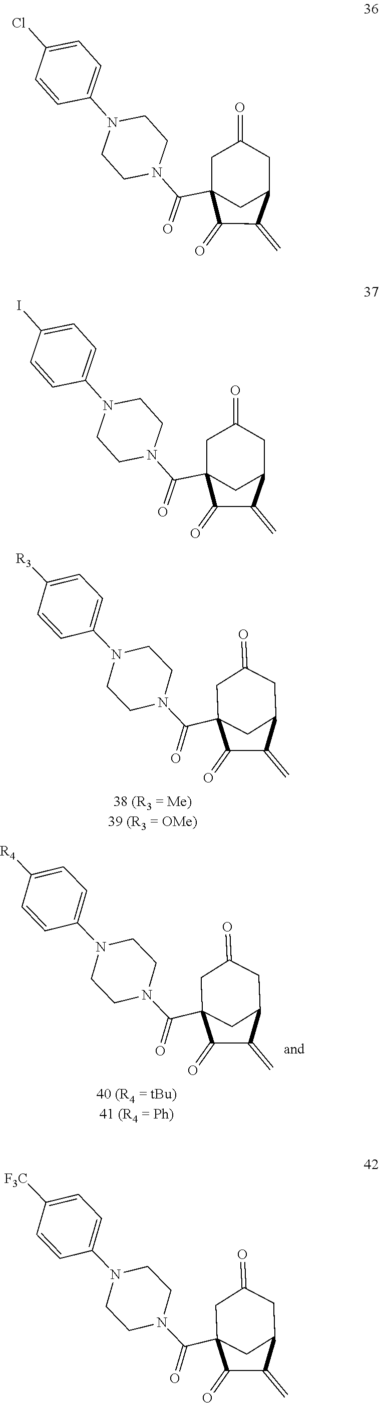
or a pharmaceutically acceptable salt, hydrate, tautomer, or optical isomer thereof.

4. A pharmaceutical composition comprising a compound of claim 1 or a pharmaceutically acceptable salt, hydrate, tautomer, or optical isomer thereof, and a pharmaceutically acceptable carrier, excipient, or diluent.

5. A pharmaceutical composition comprising (i) a compound of claim 1 or a pharmaceutically acceptable salt, hydrate, tautomer, or optical isomer thereof, (ii) one or more additional therapeutic agents, and (iii) at least one pharmaceutically acceptable carrier, excipient, or diluent.

6. A method of treating or inhibiting cancer in a patient, which method comprises administering to the patient a therapeutically effective amount of a compound of claim 1 or a pharmaceutical composition comprising the compound and a pharmaceutically acceptable carrier, excipient, or diluent.

7. The method of claim 6, wherein the cancer is leukemia or lung, breast, gastric, laryngeal, liver, or oral cancer.

8. The method of claim 6, wherein the compound can be administered orally or parenterally.

9. A method of inhibiting a Src homology region 2 containing protein tyrosine phosphatase 2- (SHP2) in a patient, which method comprises administering to the patient a therapeutically effective amount of a compound of claim 1 or a pharmaceutical composition comprising the compound and a pharmaceutically acceptable carrier, excipient, or diluent.

10. The method of claim 8, wherein the compound can be administered orally or parenterally.

11. A pharmaceutical combination comprising (i) a compound of claim 1, (ii) an additional therapeutic agent, and (iii) optionally at least one pharmaceutically acceptable carrier, excipient, or diluent.
